(19) United States
(12) Patent Application Publication
(10) Pub. No.: US 2003/0232790 A1
(43) Pub. Date: Dec. 18, 2003

(54) Dihydro-2h-napthalene-1-one inhibitors of ras farnesyl transferase

(76) Inventors: Daniele Marie Leonard, Ann Arbor, MI (US); Joseph Thomas Repine, Ann Arbor, MI (US); Gordon William Rewcastle, Auckland (NZ)

Correspondence Address: Steven R Eck, Warner Lambert Company, 2800 Plymouth Road, Ann Arbor, MI, 48105, US

(21) Appl. No.: 10/257,128

(22) PCT Filed: Apr. 17, 2001
(86) PCT No.: PCT/US01/12433

(51) Int. Cl. A61K 31/675; A61K 31/4162; A61K 31/4178; C 07D 4 5/02; C07D 233/22

(52) U.S. Cl. 514/94; 514/396; 514/397; 548/112; 548/315.4; 548/350.1

(57) ABSTRACT

The present invention provides dihydro-2H-napthalene-1-ones of formula (V), and pharmaceutically acceptable salts, esters, amides, and prodrugs thereof, which are useful for treating and preventing uncontrolled or abnormal proliferation of tissues, such as cancer, atherosclerosis, restenosis, and psoriasis. Specifically, the present invention relates to compounds that inhibit the farnesyl transferase enzyme.

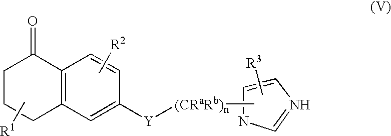

### FIELD OF THE INVENTION

The present invention relates to compounds that can be used to treat, prophylactically or otherwise, uncontrolled or abnormal proliferation of tissues. Specifically, the present invention relates to compounds that inhibit the farnesyl transferase enzyme, which has been determined to activate ras proteins that in turn activate cellular division and are implicated in cancer, restenosis, and atherosclerosis.

### SUMMARY OF THE RELATED ART

Ras protein (or p21) has been examined extensively because mutant forms are found in 20% of most types of human cancer and greater than 50% of colon and pancreatic carcinomas (Gibbs J. B., Cell, 1991;65: 1, Cartwright T. et al., Chimica. Oggi., 1992;10:26). These mutant ras proteins are deficient in the capability for feedback regulation that is present in native ras, and this deficiency is associated with their oncogenic action since the ability to stimulate normal cell division cannot be controlled by the normal endogenous regulatory cofactors. The recent discovery that the transforming activity of mutant ras is critically dependent on post-translational modifications (Gibbs J. et al., Microbiol. Rev., 1989;53:171) has unveiled an important aspect of ras function and identified novel prospects for cancer therapy.

In addition to cancel; there are other conditions of uncontrolled cellular proliferation that may be related to excessive expression and/or function of native ras proteins. Postsurgical vascular restenosis and atherosclerosis are such conditions. The use of various surgical revascularization techniques such as saphenous vein bypass grafting, endarterectomy, and transluminal coronary angioplasty are often accompanied by complications due to uncontrolled growth of neointimal tissue, known as restenosis. The biochemical causes of restenosis are poorly understood and numerous growth factors and protooncogenes have been implicated (Naftilan A. J. et al., Hypertension, 1989; 13:706 and J. Clin. Invest., 1989, 83:1419; Gibbons G. H. et al., Hypertension, 1989;14:358; Satoh T. et al., Molec. Cell. Biol., 1993;13:3706). The fact that ras proteins are known to be involved in cell division processes makes them a candidate for intervention in many situations where cells are dividing uncontrollably. In direct analogy to the inhibition of mutant ras related cancer, blockade of ras dependent processes has the potential to reduce or eliminate the inappropriate tissue proliferation associated with restenosis or atherosclerosis, particularly in those instances where normal ras expression and/or function is exaggerated by growth stimulatory factors. See, for example, Kohl et al., Nature Med., 1995;1(8):792-797.

Ras functioning is dependent upon the modification of the proteins in order to associate with the inner face of plasma membranes. Unlike other membrane-associated proteins, ras proteins lack conventional transmembrane or hydrophobic sequences and are initially synthesized in a cytosol soluble form. Ras protein membrane association is triggered by a series of post-translational processing steps that are signaled by a carboxyl terminal amino acid consensus sequence that is recognized by protein farnesyl transferase (PFT). This consensus sequence consists of a cysteine residue located four amino acids from the carboxyl terminus, followed by two lipophilic amino acids, and the C-terminal residue. The sulfhydryl group of the cysteine residue is alkylated by farnesyl pyrophosphate in a reaction that is catalyzed by protein farnesyl transferase. Following prenylation, the C-terminal three amino acids are cleaved by an endoprotease and the newly exposed alpha-carboxyl group of the prenylated cysteine is methylated by a methyl transferase.

The enzymatic processing of ras proteins that begins with farnesylation enables the protein to associate with the cell membrane. Mutational analysis of oncogenic ras proteins indicate that these post-translational modifications are essential for transforming activity. Replacement of the consensus sequence cysteine residue with other amino acids gives a ras protein that is no longer farnesylated, fails to migrate to the cell membrane, and lacks the ability to stimulate cell proliferation (Hancock J. F. et al., Cell, 1989;57: 1617; Schafer W. R. et al., Science, 1989;245:379; Casey P. J., Proc. Natl. Acad. Sci. USA, 1989;86:8323).

Recently, PFTs, also referred to as farnesyl protein transferases (FPTs), have been identified and a specific PFT from rat brain is purified to homogeneity (Reiss Y. et al., Bioch. Soc. Trans., 1992;20:487-88). The enzyme is characterized as a heterodimer composed of one alpha-subunit (49 kDa) and one beta-subunit (46 kDa), both of which are required for catalytic activity. High expression levels of mammalian PFT in a baculovirus system and purification of the recombinant enzyme in active form has also been accomplished (Chen W.-J. et al., J. Biol. Chem., 1993;268:9675).

In light of the foregoing, the discovery that the function of oncogenic ras proteins is critically dependent on their post-translational processing provides a means of cancer chemotherapy through inhibition of the processing enzymes. The identification and isolation of a PFT that catalyzes the addition of a farnesyl group to ras proteins provides a promising target for such intervention. Ras farnesyl transferase inhibitors have been shown to have anticancer activity in several recent articles.

Ras inhibitor agents act by inhibiting farnesyl transferase, the enzyme responsible for the post-translational modification of the ras protein which helps to anchor the protein product of the ras gene to the cell membrane. The role of the ras mutation in transducing growth signals within cancer cells relies on the protein being in the cell membrane. Inhibition of farnesyl transferase will result in the ras protein remaining in the cytosol and, consequently, being unable to transmit growth signals. These facts are well-known in the literature.

A peptidomimetic inhibitor of farnesyl transferase B956 and its methyl ester B1086 at 100 mg/kg have been shown to inhibit tumor growth by EJ-1 human bladder carcinoma, HT18O human fibrosarcoma, and human colon carcinoma xenografts in nude mice (Nagasu T. et al., Cancer Res., 1995;55:5310-5314). Furthermore, inhibition of tumor growth by B956 has been shown to correlate with inhibition of ras post-translational processing in the tumor. Other ras farnesyl transferase inhibitors have been shown to specifically prevent ras processing and membrane localization and are effective in reversing the transformed phenotype of mutant ras containing cells (Sepp-Lorenzino L. et al., Cancer Res., 1995;55:5302-5309).

In another report (Sun J. et al., Cancer Res., 1995;55:4243-4247), a ras farnesyl transferase inhibitor FTI276 has been shown to selectively block tumor growth in nude mice of a human lung carcinoma with K-ras mutation and p53 deletion. In yet another report, daily administration of a ras farnesyl transferase inhibitor L-744,832 caused tumor regression of mammary and salivary carcinomas in ras transgenic mice (Kohl et al., Nature Med., 1995;1(8):748-792). Thus, ras farnesyl transferase inhibitors have benefit in certain forms of cancer, particularly those dependent on oncogenic ras for their growth.

It is well-known, however, that human cancer is often manifested when several mutations in important genes occurs, one or more of which mutations may be responsible for controlling growth and metastases. A single mutation may not be enough to sustain growth but after the occurrence of only two of three mutations, tumors can develop and grow. It is difficult, therefore, to determine which of these mutations may be primarily driving the growth in a particular type of cancer. Thus, ras farnesyl transferase inhibitors can have therapeutic utility in tumors not solely dependent on oncogenic forms of ras for their growth. For example, it has been shown that various ras FT-inhibitors have antiproliferative effects in vivo against tumor lines with either wild-type or mutant ras (Sepp-Lorenzino, supra.). In addition, there are several ras-related proteins that are prenylated. Proteins such as R-Ras2/TC21 are ras-related proteins that are prenylated in vivo by both farnesyl transferase and geranylgeranyl transferase I (Carboni et al., Oncogene, 1995;10:1905-1913). Therefore, ras farnesyl transferase inhibitors could also block the prenylation of the above proteins and, therefore, would then be useful in inhibiting the growth of tumors driven by other oncogenes.

With regard to the restenosis and vascular proliferative diseases, it has been shown that inhibition of cellular ras prevents smooth muscle proliferation after vascular injury in vivo (Indolfi C. et al., Nature Med., 1995; 1(6):541-545). This report definitively supports a role for farnesyl transferase inhibitors in this disease, showing inhibition of accumulation and proliferation of vascular smooth muscle.

### SUMMARY OF THE INVENTION

This invention provides novel dihydro-2H-napthalene-1-ones that are useful for treating and preventing uncontrolled or abnormal proliferation of tissues, such as cancer, atherosclerosis, restenosis, psoriasis, and endometriosis. Specifically, the present invention relates to compounds that inhibit the farnesyl transferase enzyme. The compounds also inhibit amyloidosis, and are thus useful to treat conditions caused by amyloidosis, such as Alzheimer's disease. The compounds are readily synthesized and can be administered to mammals by a variety of routes, including orally and parenterally, and have little or no toxicity.

The present invention provides a compound of Formula V

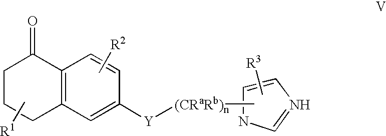

and pharmaceutically acceptable salts, esters, amides, and prodrugs thereof,

wherein:

R^{a}, R^{b}, and R^{c} are independently hydrogen, (C₁-C₆)-alkyl, (C₂-C₆)-alkenyl, aryl, heteroaryl, arylalkyl, or heteroarylalkyl, wherein the aryl, heteroaryl, arylalkyl, or heteroarylalkyl is optionally substituted with one, two, or three groups independently selected from the group consisting of alkyl, O-alkyl, S-alkyl, OH, SH, —CN, halogen, 1,3-dioxolanyl, CF₃, NO₂, NH₂, NHCH₃, N(CH₃)₂, NHCO-alkyl, —(CH₂)ₘCO₂H, —(CH₂)ₘCO₂-alkyl, —(CH₂)ₘSO₃H, —NH alkyl, —N(alkyl)₂, —(CH₂)ₘPO₃H₂, —(CH₂)ₘPO₃(alkyl)₂, —(CH₂)ₘSO₂NH₂, and —(CH₂)ₘSO₂NH-alkyl, wherein m is 0, 1, 2, or 3;

R¹ and R² are independently hydrogen, (C₁-C₆)-alkyl, (C₂-C₆)-alkenyl, aryl, heteroaryl, arylalkyl, or heteroarylalkyl, wherein the aryl, heteroaryl, arylalkyl or heteroarylalkyl is optionally substituted with one, two, or three groups independently selected from the group consisting of alkyl, O-alkyl, S-alkyl, OH, SH, —CN, halogen, 1,3-dioxolanyl, CF₃, NO₂, NH₂, NHCH₃, N(CH₃)₂, NHCO-alkyl, —(CH₂)ₘCO₂H, —(CH₂)ₘCO₂-alkyl, —(CH₂)ₘSO₃H, —NH-alkyl, —N(alkyl)₂, —(CH₂)ₘPO₃H₂, —(CH₂)ₘPO₃(alkyl)₂, (CH₂)ₘSO₂NH₂, —(CH₂)ₘ-heteroaryl, —(CH₂)ₘS-aryl, —(CH₂)ₘS-heteroaryl, —(CH₂)ₘSO₂-aryl, —(CH₂)ₘSO₂-heteroaryl, and —(CH₂)ₘSO₂NH-alkyl, wherein m is 0, 1, 2, or 3, and wherein each of the R¹ and R² groups can be attached through a linker, or through a lower alkyl optionally interrupted by a linker, said linker selected from the group consisting of

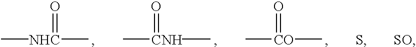

SO₂, O, and NR^{c};

Y is NR^{c}, O, —CHR^{c}, or S;

n is 0, 2, or 3, provided that when the imidazole is attached at the imidazole nitrogen to (CR^{a}R^{b})ₙ and Y is O, NR^{c} or S, then n is not 0; and

R³ is aryl, heteroarylalkyl, or arylalkyl, wherein the aryl, heteroaryl or arylalkyl is optionally substituted with up to three groups selected from the group consisting of halogen, (C₁-C₆)-alkyl, amino, (C₁-C₆)-alkoxy, hydroxy, trifluoromethyl, mono- or dialkylamino, (C₁-C₆)-thioalkoxy, cyano, nitro, 1,3-dioxolanyl, NHCO(C₁-C₆)-alkyl, (CH₂)ₘCO₂H, (CH₂)ₘCO₂(C₁-C₆)-alkyl, (CH₂)ₘSO₃H, —(CH₂)ₘPO₃H₂, (CH₂)ₘPO₃ [(C₁-C₆)-alkyl]2, (CH₂)ₘSO₂NH₂, and (CH₂)ₘSO₂NH(C₁-C₆)-alkyl, wherein m is 0, 1, 2, or 3.

The present invention also provides a compound of Formula VI

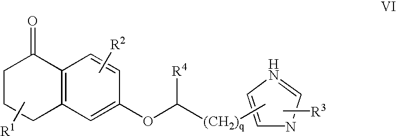

and pharmaceutically acceptable salts, esters, amides, and prodrugs thereof,

wherein:

R¹ and R² are independently hydrogen, (C₁-C₆)-alkyl, (C₂-C₆)-alkenyl, aryl, heteroaryl, arylalkyl, or heteroarylalkyl, wherein the aryl, heteroaryl, arylalkyl or heteroarylalkyl is optionally substituted with one, two, or three groups independently selected from the group consisting of alkyl, O-alkyl, S-alkyl, OH, SH, —CN, halogen, 1,3-dioxolanyl, CF₃, NO₂, NH₂, NHCH₃, N(CH₃)₂, NHCO-alkyl, —(CH₂)ₘCO₂H, —(CH₂)ₘCO₂-alkyl, —(CH₂)ₘSO₃H, —NH-alkyl, —N(alkyl)₂, —(CH₂)ₘPO₃H₂, —(CH₂)ₘPO₃(alkyl)₂, —(CH₂)ₘSO₂NH₂, —(CH₂)ₘ-heteroaryl, —(CH₂)ₘS-aryl, —(CH₂)ₘS-heteroaryl, —(CH₂)ₘSO₂-aryl, —(CH₂)ₘSO₂-heteroaryl, and —(CH₂)ₘSO₂NH-alkyl, wherein m is 0, 1, 2, or 3; and wherein each of the R¹ and R² groups can be attached through a linker, or through a lower alkyl optionally interrupted by a linker, said linker selected from the group consisting of

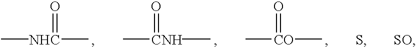

SO₂, O, and NR^{c};

R^{c} is hydrogen, (C₁-C₆)-alkyl, or aryl;

q is 1 or 2;

R⁴ is hydrogen, heteroaryl, or aryl, wherein the aryl or heteroaryl is optionally substituted with up to three groups selected from the group consisting of halogen, (C₁-C₆)-alkyl, amino, (C₁-C₆)-alkoxy, hydroxy, trifluoromethyl, mono- or dialkylamino, (C₁-C₆)-thioalkoxy, cyano, nitro, 1,3-dioxolanyl, NHCO(C₁-C₆)-alkyl, (CH₂)ₘCO₂H, (CH₂)ₘCO₂(C₁-C₆)-alkyl, (CH₂)ₘSO₃H, —(CH₂)ₘPO₃H₂, (CH₂)ₘPO₃ [(C₁-C₆)-alkyl]₂, (CH₂)ₘSO₂NH₂, and (CH₂)ₘSO₂NH(C₁-C₆)-alkyl, wherein m is 0, 1, 2, or 3; and

R³ is aryl, heteroarylalkyl, or arylalkyl, wherein the aryl, heteroaryl or arylalkyl is optionally substituted with up to three groups selected from the group consisting of halogen, (C₁-C₆)-alkyl, amino, (C₁-C₆)-alkoxy, hydroxy, trifluoromethyl, mono- or dialkylamino, (C₁-C₆)-thioalkoxy, cyano, nitro, 1,3-dioxolanyl, NHCO(C₁-C₆)-alkyl, (CH₂)ₘCO₂H, (CH₂)ₘCO₂(C₁-C₆)-alkyl, (CH₂)ₘSO₃H, —(CH₂)ₘPO₃H₂, (CH₂)ₘPO₃ [(C₁-C₆)-alkyl]₂, (CH₂)ₘSO₂NH₂, and (CH₂)ₘSO₂NH(C₁-C₆)-alkyl, wherein m is 0, 1, 2, or 3.

Additionally, the present invention provides a compound of Formula VIII

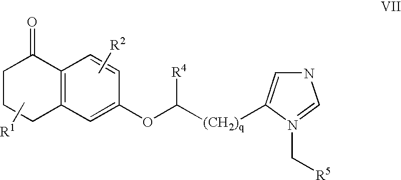

and pharmaceutically acceptable salts, esters, amides, and prodrugs thereof,

wherein:

R¹ and R² are independently hydrogen, (C₁-C₆)-alkyl, (C₂-C₆)-alkenyl, aryl, heteroaryl, arylalkyl, or heteroarylalkyl, wherein the aryl, heteroaryl, arylalkyl or heteroarylalkyl is optionally substituted with one, two, or three groups independently selected from the group consisting of alkyl, O-alkyl, S-alkyl, OH, SH, —CN, halogen, 1,3-dioxolanyl, CF₃, NO₂, NH₂, NHCH₃, N(CH₃)₂, NHCO-alkyl, —(CH₂)ₘCO₂H, —(CH₂)ₘCO₂-alkyl, —(CH₂)ₘSO₃H, —NH-alkyl, —N(alkyl)₂, —(CH₂)ₘPO₃H₂, —(CH₂)ₘPO₃(alkyl)₂, —(CH₂)ₘSO₂NH₂, —(CH₂)ₘ-heteroaryl, —(CH₂)ₘS-aryl, —(CH₂)ₘS-heteroaryl, —(CH₂)ₘSO₂-aryl, —(CH₂)ₘSO₂-heteroaryl, and —(CH₂)ₘSO₂NH-alkyl, wherein m is 0, 1, 2, or 3; and wherein each of the R¹ and R² groups can be attached through a linker; or through a lower alkyl optionally interrupted by a linker, said linker selected from the group consisting of

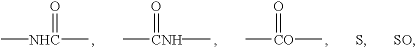

SO₂, O, and NR^{c};

R^{c} is hydrogen, (C₁-C₆)-alkyl, or aryl;

q is 1 or 2;

R⁴ is hydrogen, heteroaryl, or aryl, wherein the aryl or heteroaryl is optionally substituted with up to three groups selected from the group consisting of halogen, (C₁-C₆)-alkyl, amino, (C₁-C₆)-alkoxy, hydroxy, trifluoromethyl, mono- or dialkylamino, (C₁-C₆)-thioalkoxy, cyano, nitro, 1,3-dioxolanyl, NHCO(C₁-C₆)-alkyl, (CH₂)ₘCO₂H, (CH₂)ₘCO₂(C₁-C₆)-alkyl, (CH₂)ₘSO₃H, —(CH₂)ₘPO₃H₂, (CH₂)ₘPO₃ [(C₁-C₆)-alkyl]₂, (CH₂)ₘSO₂NH₂, and (CH₂)ₘSO₂NH(C₁-C₆)-alkyl, wherein m is 0, 1, 2, or 3; and

R⁵ is aryl optionally substituted with up to three groups selected from the group consisting of halogen, (C₁-C₆)-alkyl, amino, (C₁-C₆)-alkoxy, hydroxy, trifluoromethyl, mono- or dialkylamino, (C₁-C₆)-thioalkoxy, cyano, nitro, 1,3-dioxolanyl, NHCO(C₁-C₆)-alkyl, (CH₂)ₘCO₂H, (CH₂)ₘCO₂(C₁-C₆)-alkyl, (CH₂)ₘSO₃H, —(CH₂)ₘPO₃H₂, (CH₂)ₘPO₃ [(C₁-C₆)-alkyl]₂, (CH₂)ₘSO₂NH₂, and (CH₂)ₘSO₂NH(C₁-C₆)-alkyl, wherein m is 0, 1, 2, or 3.

Furthermore, the present invention provides a compound of Formula VIII

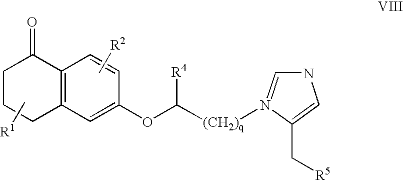

and pharmaceutically acceptable salts, esters, amides, and prodrugs thereof,

wherein:

R¹ and R² are independently hydrogen, (C₁-C₆)-alkyl, (C₂-C₆)-alkenyl, aryl, heteroaryl, arylalkyl, or heteroarylalkyl, wherein the aryl, heteroaryl, arylalkyl or heteroarylalkyl is optionally substituted with one, two, or three groups independently selected from the group consisting of alkyl, O-alkyl, S-alkyl, OH, SH, —CN, halogen, 1,3-dioxolanyl, CF₃, NO₂, NH₂, NHCH₃, N(CH₃)₂, NHCO-alkyl, —(CH₂)ₘCO₂H, —(CH₂)ₘCO₂-alkyl, —(CH₂)ₘSO₃H, —NH-alkyl, —N(alkyl)₂, —(CH₂)ₘPO₃H₂, —(CH₂)ₘPO₃(alkyl)₂, —(CH₂)ₘSO₂NH₂, —(CH₂)ₘ-heteroaryl, —(CH₂)ₘS-aryl, —(CH₂)ₘS-heteroaryl, —(CH₂)ₘSO₂-aryl, —(CH₂)ₘSO₂-heteroaryl, and —(CH₂)ₘSO₂NH-alkyl, wherein m is 0, 1, 2, or 3; and wherein each of the R¹ and R² groups can be attached through a linker, or through a lower alkyl optionally interrupted by a linker, said linker selected from the group consisting of

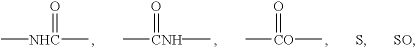

SO₂, O, and NR^{c};

R^{c} is hydrogen, (C₁-C₆)-alkyl, or aryl;

q is 1 or 2;

R⁴ is hydrogen, heteroaryl, or aryl, wherein the aryl or heteroaryl is optionally substituted with up to three groups selected from the group consisting of halogen, (C₁-C₆)-alkyl, amino, (C₁-C₆)-alkoxy, hydroxy, trifluoromethyl, mono- or dialkylamino, (C₁-C₆)-thioalkoxy, cyano, nitro, 1,3-dioxolanyl, NHCO(C₁-C₆)-alkyl, (CH₂)ₘCO₂H, (CH₂)ₘCO₂(C₁-C₆)-alkyl, (CH₂)ₘSO₃H, —(CH₂)ₘPO₃H₂, (CH₂)ₘPO₃ [(C₁-C₆)-alkyl]₂, (CH₂)ₘSO₂NH₂, and (CH₂)ₘSO₂NH(C₁-C₆)-alkyl, wherein m is 0, 1, 2, or 3; and

R⁵ is aryl optionally substituted with up to three groups selected from the group consisting of halogen, (C₁-C₆)-alkyl, amino, (C₁-C₆)-alkoxy, hydroxy, trifluoromethyl, mono- or dialkylamino, (C₁-C₆)-thioalkoxy, cyano, nitro, 1,3-dioxolanyl, NHCO(C₁-C₆)-alkyl, (CH₂)ₘCO₂H, (CH₂)ₘCO₂(C₁-C₆)-alkyl, (CH₂)ₘSO₃H, —(CH₂)ₘPO₃H₂, (CH₂)ₘPO₃ [(C₁-C₆)-alkyl]2, (CH₂)ₘSO₂NH₂, and (CH₂)ₘSO₂NH(C₁-C₆)-alkyl, wherein m is 0, 1, 2, or 3.

The present invention also provides a compound of Formula IX

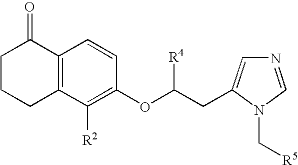

and pharmaceutically acceptable salts, esters, amides, and prodrugs thereof,

wherein:

R² is hydrogen, (C₁-C₆)-alkyl, aryl, heteroaryl, arylalkyl, or heteroarylalkyl, wherein the aryl, heteroaryl, arylalkyl, or heteroarylalkyl is optionally substituted with a group independently selected from the group consisting of alkyl, O-allyl, S-alkyl, OH, SH, —CN, halogen, 1,3-dioxolanyl, CF₃, NO₂, NH₂, NHCH₃, N(CH₃)₂, NHCO-alkyl, —(CH₂)ₘCO₂H, —(CH₂)ₘCO₂-alkyl, —(CH₂)ₘSO₃H, —NH-alkyl, —N(alkyl)₂, —(CH₂)ₘPO₃H₂, —(CH₂)ₘPO₃ (alkyl)₂, (CH₂)ₘSO₂NH₂, —(CH₂)ₘ-heteroaryl, —(CH₂)ₘS-aryl, —(CH₂)ₘS-heteroaryl, —(CH₂)ₘSO₂-aryl, —(CH₂)ₘSO₂-heteroaryl, and —(CH₂)ₘSO₂NH-alkyl, wherein m is 0, 1, 2, or 3, and wherein each of the R¹ and R² groups can be attached through a linker, or through a lower alkyl optionally interrupted by a linker, said linker selected from the group consisting of

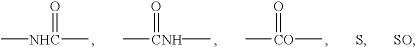

SO₂, O, and NH;

R⁴ is hydrogen or phenyl; and

R⁵ is aryl optionally substituted by (C₁-C₆)-alkyl, (C₁-C₆)-alkoxy, or cyano.

Additionally, the present invention provides a compound of Formula X

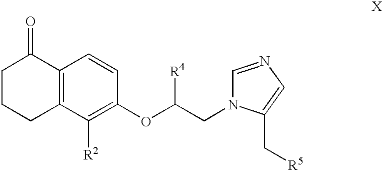

and pharmaceutically acceptable salts, esters, amides, and prodrugs thereof,

wherein:

R² is hydrogen, (C₁-C₆)-alkyl, aryl, heteroaryl, arylalkyl, or heteroarylalkyl, wherein the aryl, heteroaryl, arylalkyl, or heteroarylalkyl is optionally substituted with a group independently selected from the group consisting of alkyl, O-alkyl, S-alkyl, OH, SH, —CN, halogen, 1,3-dioxolanyl, CF₃, NO₂, NH₂, NHCH₃, N(CH₃)₂, NHCO-alkyl, —(CH₂)ₘCO₂H, —(CH₂)ₘCO₂-alkyl, —(CH₂)ₘSO₃H, —NH-alkyl, —N(alkyl)₂, —(CH₂)ₘPO₃H₂, —(CH₂)ₘPO₃(alkyl)₂, —(CH₂)ₘSO₂NH₂, —(CH₂)ₘ-heteroaryl, —(CH₂)ₘS-aryl, —(CH₂)ₘS-heteroaryl, —(CH₂)ₘSO₂-aryl, —(CH₂)ₘSO₂-heteroaryl, and —(CH₂)ₘSO₂NH-alkyl, wherein m is 0, 1, 2, or 3, and wherein each of the R¹ and R² groups can be attached through a linker, or through a lower alkyl optionally interrupted by a linker, said linker selected from the group consisting of

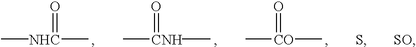

SO₂, O, and NH;

R⁴ is hydrogen or phenyl; and

R⁵ is aryl optionally substituted by (C₁-C₆)-alkyl, (C₁-C₆)-alkoxy, or cyano.

The present invention also provides a pharmaceutically acceptable composition that comprises a compound of Formulas I-X and a pharmaceutically acceptable carrier. Additionally, the present invention provides a pharmaceutical composition comprising a compound of Formulas I-X and a pharmaceutically acceptable carrier, excipient or diluent.

The present invention also provides a method of treating or preventing restenosis, the method comprising administering to a patient having restenosis or at risk of having restenosis a therapeutically effective amount of a compound of Formulas I-X.

The present invention also provides a method of treating cancer, the method comprising administering to a patient having cancer a therapeutically effective amount of a compound of Formulas I-X. In a preferred embodiment of the method of treating cancer, the cancer is lung, colon, pancreatic, thyroid, or bladder cancer.

The present invention also provides a method of treating atherosclerosis, the method comprising administering to a patient having atherosclerosis a therapeutically effective amount of a compound of Formulas I-X.

Also provided is a method of treating or preventing restenosis or atherosclerosis or treating cancer, the method of comprising administering to a patient having restenosis or atherosclerosis, or at risk of having restenosis or atherosclerosis, or having cancer a therapeutically effective amount of a compound of Formulas I-X.

Furthermore, the present invention provides a use of a compound of Formulas I-X, or a pharmaceutically acceptable salt thereof for the manufacture of a medicament for treating or preventing any of the diseases or disease states mentioned above.

### DETAILED DESCRIPTION OF THE INVENTION

The novel compounds encompassed by the instant invention are those described by the general Formulas V-X set forth above, and the pharmaceutically acceptable salts, esters, amides, and prodrugs thereof.

In addition to the compounds of Formulas V-X, the present invention encompasses compounds of Formulas I-IV. The compounds of the invention are members of the class of compounds of Formula I:

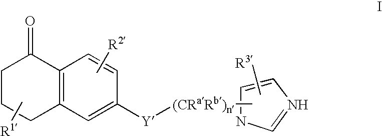

wherein:

R^{a′}, R^{b′}, and R^{c′} independently are hydrogen, (C₁-C₆)-alkyl, (C₂-C₆)-alkenyl, aryl, substituted aryl, heteroaryl, substituted heteroaryl, arylalkyl, substituted arylalkyl, heteroarylalkyl, or substituted heteroarylalkyl; or

R^{1′} and R^{2′} independently are hydrogen, (C₁-C₆)-alkyl, (C₂-C₆)-alkenyl, aryl, heteroaryl, substituted heteroaryl, arylalkyl, substituted arylalkyl, heteroarylalkyl, or substituted heteroarylalkyl, wherein each of the foregoing groups may be attached directly through a linker, or through a lower alkyl group, said alkyl group optionally being interrupted by a linker, said linker selected from

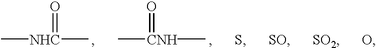

and NR^{c′};

n′ is 0, 2, or 3, provided that when the imidazole is attached at the imidazole nitrogen to (CR^{a′}R^{b′})_{n′} and Y′ is O, NR^{c′}, or S, then n is not 0;

Y′ is NR^{c′}, O, CHR^{c′}, or S; and

R^{3′} is aryl, heteroarylalkyl, or arylalkyl where each ring is optionally substituted independently with up to three groups selected from halogen, (C₁-C₆)-alkyl, amino, (C₁-C₆)-alkoxy, hydroxy, trifluoromethyl, mono- or dialkylamino, (C₁-C₆)-thioalkoxy, cyano, nitro, 1,3-dioxolanyl, NHCO (C₁-C₆)-allyl, (CH₂)_{m′}CO₂H, (CH₂)_{m′}CO₂ (C₁-C₆)-alkyl, (CH₂)_{m′}SO₃H, —(CH₂)_{m′}PO₃H₂, (CH₂)_{m′}PO₃ (C₁-C₆)-(alkyl)₂, (CH₂)_{m′}SO₂NH₂, and (CH₂)_{m′}SO₂NH(C₁-C₆)-alkyl wherein m′is 0, 1, 2, or 3.

Preferred compounds of Formula I are those where R^{1′} is hydrogen; R^{2′} is hydrogen, lower alkyl, arylalkyl, arylaminoalkyl, arylamino, arylcarbonylamino, alkoxyalkyl, phenylsulfonylalkyl, or alkoxycarbonylallyl; Y′ is O; R^{a′} is hydrogen; R^{b′} is hydrogen, aryl or substituted aryl; n′ is 2; and R^{3′} is benzyl or substituted benzyl.

In addition to the compounds of Formula I, the invention encompasses compounds of Formula II:

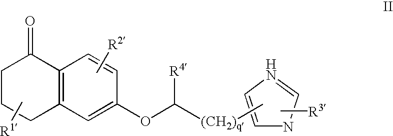

wherein R^{1′}, R^{2′}, and R^{3′} are as defined above for Formula I; q′ is 1 or 2; and R^{4′} is hydrogen, aryl, heteroaryl, or substituted aryl.

Preferred compounds of Formula II are those in which R^{1′} is hydrogen; R^{2′} is hydrogen, lower alkyl, arylalkyl, arylaminoalkyl, arylamino, arylcarbonylamino, alkoxyalkyl, phenylsulfonylalkyl, or alkoxycarbonylalkyl; q′ is 1; R^{4′} is hydrogen, pyridyl, or phenyl; and R^{3′} is benzyl or substituted benzyl.

More preferred compounds of Formula II are where R^{2′} is at the 5-position.

In addition to the compounds of Formula I, the invention encompasses compounds of Formula III:

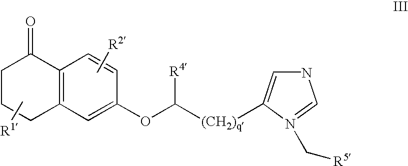

wherein q′, R^{1′}, R^{2′}, and R^{4′} are as defined above for Formulas I and II, and R^{5′} is aryl or substituted aryl. Preferred compounds of Formula III are where q′ is 1; R^{1′} is hydrogen; R^{2′} is hydrogen, lower alkyl, lower alkyl-sulfonylalkyl, arylalkyl, arylaminoalkyl, arylamino, arylcarbonylamino, alkoxyalkyl, phenylsulfonylalkyl, heteroarylsulfonylalkyl, or alkoxycarbonylalkyl; R^{4′} is hydrogen, pyridyl, or phenyl; and R^{5′} is phenyl or substituted phenyl. More preferred compounds of Formula III are where R^{2′} is at the 5-position.

In addition to the compounds of Formula I, the invention encompasses compounds of Formula IV:

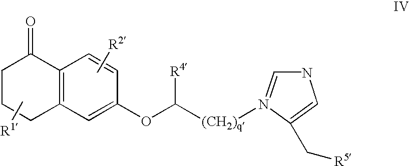

wherein q′, R^{1′}, R^{2′}, and R^{4′} are as defined above for Formulas I and II, and R^{5′} is aryl or substituted aryl.

Preferred compounds of Formula IV are where q′ is 1; R^{1′} is hydrogen; R^{2′} is hydrogen, lower alkyl, lower alkyl-sulfonylalkyl, arylalkyl, heteroarylalkyl, arylaminoalkyl, arylamino, arylcarbonylamino, alkoxyalkyl, phenylsulfonylalkyl, heteroarylsulfonylalkyl, or alkoxycarbonylalkyl; R^{4′} is hydrogen, pyridyl, or phenyl; and R^{5′} is phenyl or substituted phenyl.

More preferred compounds of Formula IV are where R^{2′} is at the 5-position.

The terms “alkyl,” “lower alkyl,” or “(C₁-C₆)-alkyl” mean a straight or branched hydrocarbon having from 1 to 6 carbon atoms and includes, for example, methyl, ethyl, n-propyl, isopropyl, n-butyl, sec-butyl, isobutyl, tert-butyl, n-pentyl, n-hexyl, and the like. The alkyl group can also be substituted with one or more of the substituents listed below for aryl.

The terms “alkenyl,” “lower alkenyl,” or “(C₂-C₆)-alkenyl” mean a straight or branched hydrocarbon having from 2 to 6 carbon atoms and 1 or 2 double bonds, and includes, for example, allyl, 3-methyl-but-2-enyl, 2-methyl-but-2-enyl, geranyl, and the like. The term “(C₂-C₆)-alkenyl” includes within its definition the term “(C₂-C₄)-alkenyl”. The alkenyl group can also be substituted with one or more of the substituents listed below for aryl.

The term “cycloalkyl” means a saturated hydrocarbon ring which contains from 3 to 7 carbon atoms, for example, cyclopropyl, cyclobutyl, cyclopentyl, cyclohexyl, adamantyl, and the like.

By “alkoxy,” “lower alkoxy,” or “(C₁-C₆)-alkoxy” in the present invention is meant straight or branched chain alkoxy groups having 1 to 6 carbon atoms, such as, for example, methoxy, ethoxy, propoxy, isopropoxy, n-butoxy, sec-butoxy, tert-butoxy, pentoxy, 2-pentyl, isopentoxy, neopentoxy, hexoxy, 2-hexoxy, 3-hexoxy, and 3-methylpentoxy.

The term “aryl” means an unsubstituted aromatic carbocyclic group having a single ring (e.g., phenyl), multiple rings (e.g., biphenyl), or multiple condensed rings in which at least one is aromatic (e.g., 1,2,3,4-tetrahydronaphthyl, naphthyl, anthryl, or phenanthryl). The term “substituted aryl” means an aryl substituted by 1 to 3 substituents selected from alkyl, O-alkyl, S-alkyl, OH, SH, —CN, halogen, 1,3-dioxolanyl, CF₃, NO₂, NH₂, NHCH₃, N(CH₃)₂, NHCO-alkyl, —(CH₂)ₘCO₂H, —(CH₂)ₘCO₂-alkyl, —(CH₂)ₘSO₃H, —NH-alkyl, —N(alkyl)₂, —(CH₂)ₘPO₃H₂, —(CH₂)ₘPO₃(alkyl)₂, —(CH₂)ₘSO₂NH₂, —(CH₂)ₘ-heteroaryl, —(CH₂)ₘS-aryl, —(CH₂)ₘS-heteroaryl, —(CH₂)ₘSO₂-aryl, —(CH₂)ₘSO₂-heteroaryl, and —(CH₂)ₘSO₂NH-alkyl, wherein alkyl is defined as above, and m is 0, 1, 2, or 3.

The term “arylalkyl” means an alkyl moiety (as defined above) substituted with an aryl moiety (also as defined above). An arylalkyl may be substituted (“substituted arylalkyl”) by 1 to 3 substituents selected from the group as defined above for “substituted aryl.”

By halogen, in the present invention, is meant fluorine, bromine, chlorine, and iodine.

By heteroaryl (aromatic heterocycle) in the present invention is meant one or more aromatic ring systems of 5-, 6-, or 7-membered rings containing at least one and up to four heteroatoms selected from nitrogen, oxygen, or sulfur. Such heteroaryl groups include, for example, thienyl, furanyl, thiazolyl, imidazolyl, (is)oxazolyl, pyridyl, pyrimidinyl, (iso)quinolinyl, naphthyridinyl, benzimidazolyl, and benzoxazolyl. The term “substituted heterocycle” means a heterocycle substituted by 1 to 3 substituents selected from the group as defined above for “substituted aryl.”

The term “heteroarylalkyl” means an alkyl moiety (as defined above) substituted with a heteroaryl moiety (also as defined above). An heteroarylalkyl may be substituted (“substituted heteroarylallyl”) by 1 to 3 substituents selected from the group as defined above for “substituted aryl.”

The symbol “—” means a bond.

The following abbreviations are used in the application.

| | |
|---|---|
| HPLC | High pressure liquid chromatography |
| mp | Melting point |
| THF | Tetrahydrofuran |
| APCI | Atmospheric pressure chemical ionization |
| MS | Mass spectrometiy |
| DMF | N,N′-Dimethylformamide |
| Et₃N | Triethylamine |
| FABHRMS | Fast atom bombardment, high resolution mass spectrometry |

The groups R¹ and R² can be attached to the dihydro naphthalenone ring system directly through a linker, or through a lower alkyl group, said alkyl being optionally interrupted by a linker selected from

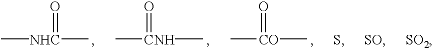

O and NR^{c}. Examples of such groups are

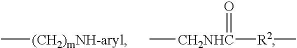

(CH₂)ₘ SO₂-alkyl, —(CH₂)ₘ—SO₂-phenyl, —CH₂CH₂—SO₂ iPr,

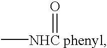

—NHCO-aryl-NH pyridyl, CH₂O-alkyl, —(CH₂)ₘ CO₂-alkyl, and CH₂O-thiazolyl, wherein m is 0, 1, 2 or 3.

The term “patient” means all animals, preferably mammals, including humans. Examples of patients include humans, cows, dogs, cats, goats, sheep, and pigs.

The term “treating” for purposes of the present invention refers to prophylaxis or prevention, amelioration or elimination of a named condition once the condition has been established.

A “therapeutically effective amount” is an amount of a compound of the present invention that when administered to a patient ameliorates or treats a symptom of restenosis, cancer, or atherosclerosis, or prevents restenosis or atherosclerosis. A therapeutically effective amount of a compound of the present invention can be easily determined by one skilled in the art by administering a quantity of a compound to a patient and observing the result. In addition, those skilled in the art are familiar with identifying patients having cancer, restenosis, or atherosclerosis or who are at risk of having restenosis.

The term “cancer” includes, but is not limited to, the following cancers: breast, ovary, uterine, fallopian tubes, endometrium, vagina, vulva, cervix, prostate, testis, penis, esophagus, glioblastoma, neuroblastoma, stomach, skin, keratoacanthoma, cutaneous or intraocular melanoma, lung, endocrine system, thyroid gland, parathyroid gland, adrenal gland, sarcoma of soft tissue, epidermoid carcinoma, large cell carcinoma, adenocarcinoma, bone, colon, adenocarcinoma, adenoma, pancreas, adenocarcinoma, thyroid, follicular carcinoma, undifferentiated carcinoma, papillary carcinoma, seminoma, melanoma, sarcoma, bladder carcinoma, liver carcinoma and biliary passages, kidney carcinoma, urethra, renal cell carcinoma, carcinoma of the renal pelvis, myeloid disorders, lymphoid disorders, Hodgkins, hairy cells, buccal cavity and pharynx (oral), lip, tongue, mouth, pharynx, small intestine, colon-rectum, cancer of the anal region, large intestine, rectum, cancer of the head or neck, brain and central nervous system, neoplasms of the central nervous system (CNS), primary CNS lymphona, spinal axis tumors, brain stem glioma, pituitary adenoma, chronic or acute leukemia, and lymphocytic lymphomas.

The term “pharmaceutically acceptable salts, esters, amides, and prodrugs” as used herein refers to those carboxylate salts, amino acid addition salts, esters, amides, and prodrugs of the compounds of the present invention which are, within the scope of sound medical judgment, suitable for use in contact with the tissues of patients without undue toxicity, irritation, allergic response, and the like, commensurate with a reasonable benefit/risk ratio, and effective for their intended use, as well as the zwitterionic forms, where possible, of the compounds of the invention. The term “salts” refers to the relatively non-toxic, inorganic, and organic acid addition salts of compounds of the present invention. These salts can be prepared in situ during the final isolation and purification of the compounds or by separately reacting the purified compound in its free base form with a suitable organic or inorganic acid and isolating the salt thus formed. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate and laurylsulphonate salts, and the like. These may include cations based on the alkali and alkaline earth metals, such as sodium, lithium, potassium, calcium, magnesium and the like, as well as non-toxic ammonium, quaternary ammonium, and amine cations including, but not limited to ammonium, tetramethylammonium, tetraethylammonium, methylamine, dimethylamine, trimethylamine, triethylamine, ethylamine, and the like. (See, for example, Berge S. M. et al., “Pharmaceutical Salts,” J. Pharm. Sci., 1977;66:1-19 which is incorporated herein by reference.)

Examples of pharmaceutically acceptable, nontoxic esters of the compounds of this invention include C₁-C₆ alkyl esters wherein the alkyl group is a straight or branched chain. Acceptable esters also include C₅-C₇ cycloalkyl esters as well as arylalkyl esters such as, but not limited to benzyl. C₁-C₄ alkyl esters are preferred. Esters of the compounds of the present invention may be prepared according to conventional methods.

Examples of pharmaceutically acceptable, non-toxic amides of the compounds of this invention include amides derived from ammonia, primary C₁-C₆ alkyl amines, and secondary C₁-C₆ dialkyl amines wherein the alkyl groups are straight or branched chain. In the case of secondary amines, the amine may also be in the form of a 5- or 6-membered heterocycle containing one nitrogen atom. Amides derived from ammonia, C₁-C₃ alkyl primary amines, and C₁-C₂ dialkyl secondary amines are preferred. Amides of the compounds of the invention may be prepared according to conventional methods.

The term “prodrug” refers to compounds that are rapidly transformed in vivo to yield the parent compound of the above formulae, for example, by hydrolysis in blood. A thorough discussion is provided in T. Higuchi and V. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987, both of which are hereby incorporated by reference.

Representative compounds of the invention are shown below in Table 1.

TABLE 1

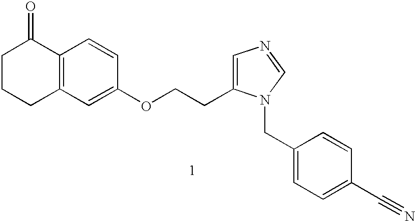

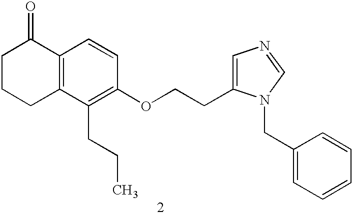

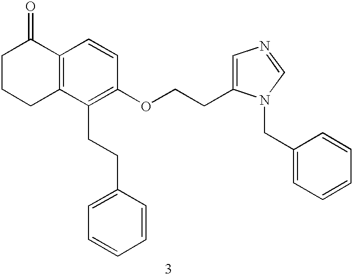

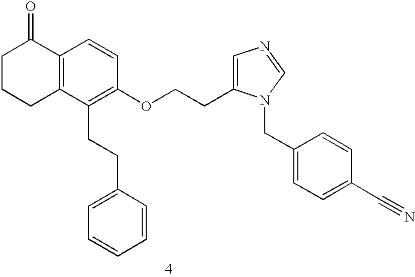

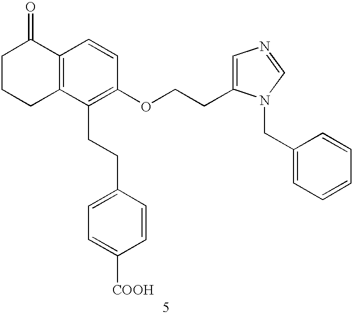

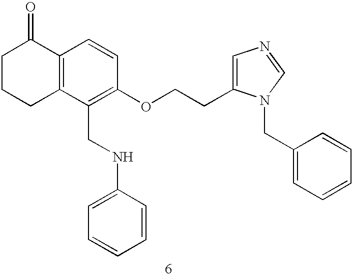

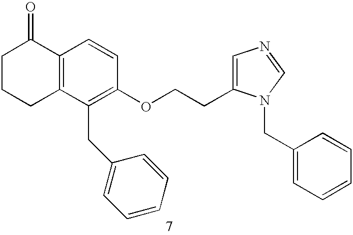

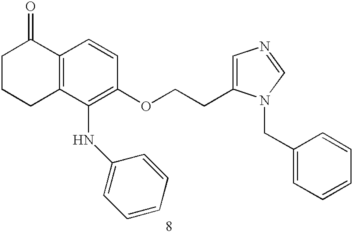

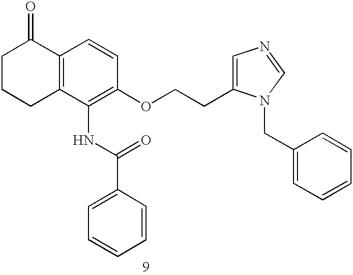

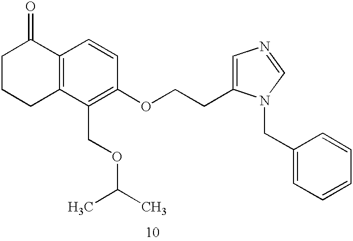

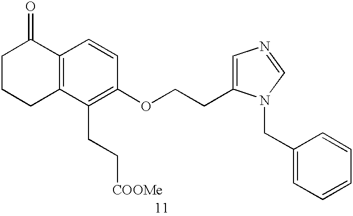

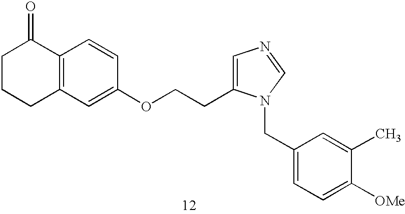

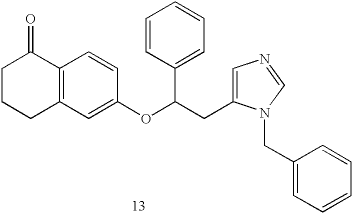

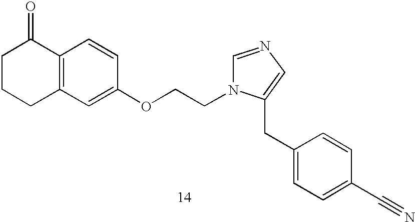

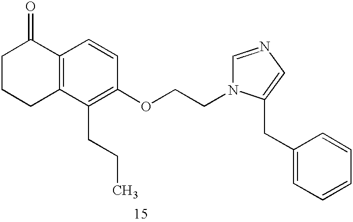

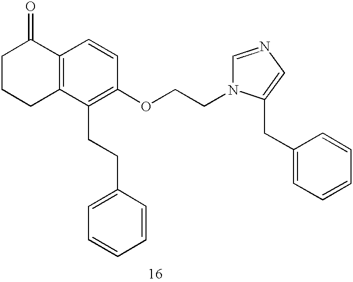

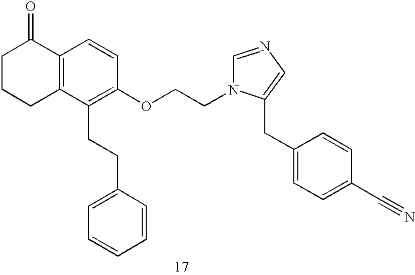

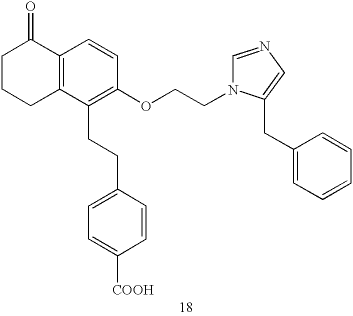

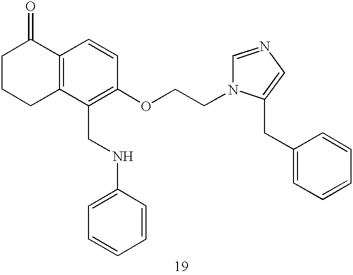

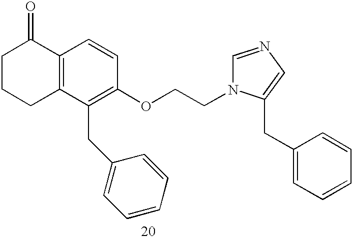

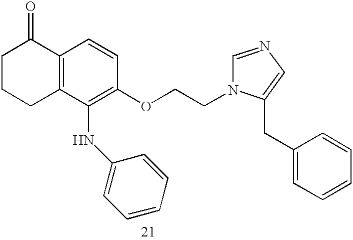

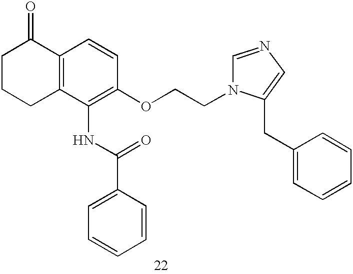

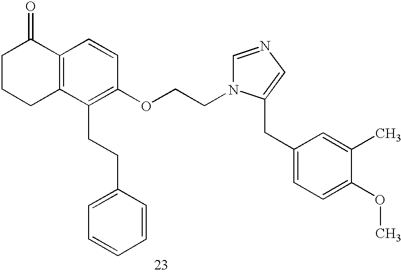

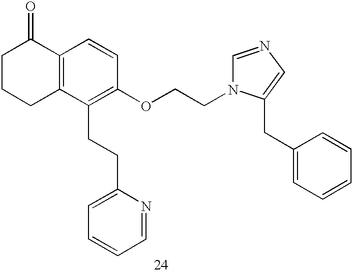

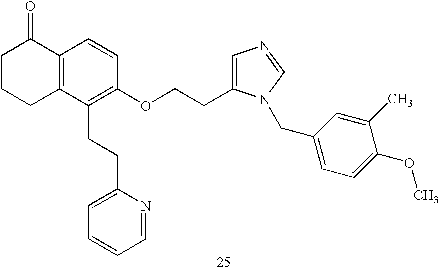

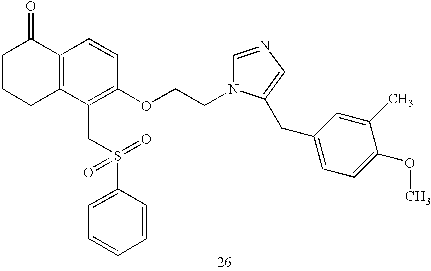

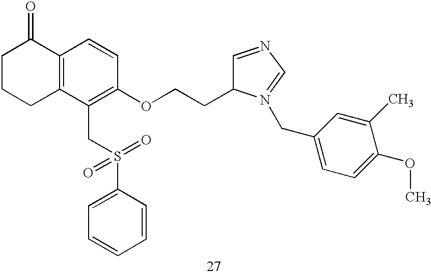

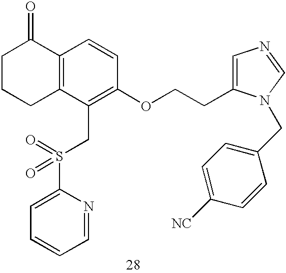

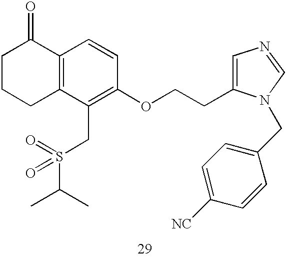

Representative compounds of the present invention, which are encompassed by Formulas I-X, and preferably Formulas V-X, include but are not limited to the compounds in Table 1 and their pharmaceutically acceptable acid or base addition salts, or amide, or prodrugs thereof.

Preferred compounds of Formula V are those wherein R¹ is hydrogen; R² is hydrogen, lower alkyl, arylalkyl, arylaminoalkyl, arylamino, arylcarbonylamino, alkoxyalkyl, or alkoxycarbonylalkyl; Y is 0; n is 2; R^{a} and R^{b} are hydrogen; R^{c} is hydrogen; and R³ is arylalkyl.

Preferred compounds of Formula V are 4-({5-[2-({5-Oxo-1-[(2-pyridinylsulfonyl)methyl]-5,6,7,8-tetrahydro-2-naphthalenyl}oxy)ethyl]-1H-imidazol-1-yl}methyl)-benzonitrile and 4-({5-[2-({1 [(Isopropylsulfonyl)methyl]-5-oxo-5,6,7,8-tetrahydro-2-naphthalenyl}oxy)ethyl]-1H-imidazol-1-yl}methyl)benzonitrile.

Preferred compounds of Formulas IX and X are those wherein R² is hydrogen, (C₁-C₆)-alkyl, aryl, heteroaryl, arylalkyl, heteroarylalkyl, or the arylalkyl is substituted with —(CH₂)ₘCO₂H; the linker is selected from the group consisting of —NHCO, —CO₂, SO₂, O, and —NH and R² is (C₁-C₆)-alkyl, aryl, or heteroaryl; and R⁴ is hydrogen.

The compounds of the present invention can be administered to a patient alone or as part of a composition that contains other components such as excipients, diluents, and carriers, such as pharamaceutically acceptable excipients, diluents, and carriers, all of which are well-known in the art. The compositions can be administered to humans and animals either orally, rectally, parenterally (intravenously, intramuscularly, or subcutaneously), intracisternally, intravaginally, intraperitoneally, intravesically, locally (powders, ointments, or drops), or as a buccal or nasal spray.

Compositions suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents, or vehicles include water, ethanol, polyols (propyleneglycol, polyethyleneglycol, glycerol, and the like), suitable mixtures thereof, vegetable oils (such as olive oil), and injectable organic esters such as ethyl oleate. Proper fluidity can be maintained, for example, by the use of a coating such as lecithin, by the maintenance of the required particle size in the case of dispersions, and by the use of surfactants.

These compositions may also contain adjuvants such as preserving, wetting, emulsifying, and dispensing agents. Prevention of the action of microorganisms can be ensured by various antibacterial and antifungal agents, for example, parabens, chlorobutanol, phenol, sorbic acid, and the like. It may also be desirable to include isotonic agents, for example sugars, sodium chloride, and the like. Prolonged absorption of the injectable pharmaceutical form can be brought about by the use of agents delaying absorption, for example, aluminum monostearate, and gelatin.

Solid dosage forms for oral administration include capsules, tablets, pills, powders, and granules. In such solid dosage forms, the active compound is admixed with at least one inert customary excipient (or carrier) such as sodium citrate or dicalcium phosphate or (a) fillers or extenders, as for example, starches, lactose, sucrose, glucose, mannitol, and silicic acid; (b) binders, as for example, carboxymethylcellulose, alignates, gelatin, polyvinylpyrrolidone, sucrose, and acacia; (c) humectants, as for example, glycerol; (d) disintegrating agents, as for example, agar-agar, calcium carbonate, potato or tapioca starch, alginic acid, certain complex silicates, and sodium carbonate; (e) solution retarders, as for example paraffin; (f) absorption accelerators, as for example, quaternary ammonium compounds; (g) wetting agents, as for example, cetyl alcohol and glycerol monostearate; (h) adsorbents, as for example, kaolin and bentonite; and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Solid compositions of a similar type may also be employed as fillers in soft- and hard-filled gelatin capsules using such excipients as lactose or milk sugar as well as high molecular weight polyethyleneglycols, and the like.

Solid dosage forms such as tablets, dragees, capsules, pills, and granules can be prepared with coatings and shells, such as enteric coatings and others well-known in the art. They may contain opacifying agents, and can also be of such composition that they release the active compound or compounds in a certain part of the intestinal tract in a delayed manner. Examples of embedding compositions which can be used are polymeric substances and waxes. The active compounds can also be in micro-encapsulated form, if appropriate, with one or more of the above-mentioned excipients.

Liquid dosage forms for oral administration include pharmaceutically acceptable emulsions, solutions, suspensions, syrups, and elixirs. In addition to the active compounds, the liquid dosage forms may contain inert diluents commonly used in the art, such as water or other solvents, solubilizing agents and emulsifiers, as for example, ethyl alcohol, isopropyl alcohol, ethyl carbonate, ethyl acetate, benzyl alcohol, benzyl benzoate, propyleneglycol, 1,3-butyleneglycol, dimethylformamide; oils, in particular, cottonseed oil, groundnut oil, corn germ oil, olive oil, castor oil, and sesame oil; glycerol, tetrahydrofurfuryl alcohol, polyethyleneglycols and fatty acid esters of sorbitan or mixtures of these substances, cremophor and the like.

Besides such inert diluents, the composition can also include adjuvants, such as wetting agents, emulsifying and suspending agents, sweetening, flavoring, and perfuming agents.

Suspensions, in addition to the active compounds, may contain suspending agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

Compositions for rectal administrations are preferably suppositories which can be prepared by mixing the compounds of the present invention with suitable non-irritating excipients or carriers such as cocoa butter, polyethyleneglycol, or a suppository wax, which are solid at ordinary temperatures but liquid at body temperature and therefore, melt in the rectum or vaginal cavity and release the active component.

Dosage forms for topical administration of a compound of this invention include ointments, powders, sprays, and inhalants. The active component is admixed under sterile conditions with a physiologically acceptable carrier and any preservatives, buffers, or propellants as may be required. Ophthalmic formulations, eye ointments, powders, and solutions are also contemplated as being within the scope of this invention.

The compounds of the present invention can be administered to a patient at dosage levels in the range of about 0.1 to about 2,000 mg per day. For a normal human adult having a body weight of about 70 kilograms, a dosage in the range of about 0.01 to about 100 mg per kilogram of body weight per day is preferable. The specific dosage used, however, can vary. For example, the dosage can depend on a numbers of factors including the requirements of the patient, the severity of the condition being treated, and the pharmacological activity of the compound being used. The determination of optimum dosages for a particular patient is well known to those skilled in the art.

The compounds of the present invention can exist in different stereoisomeric forms by virtue of the presence of asymmetric centers in the compounds. It is contemplated that all stereoisomeric forms of the compounds as well as mixtures thereof, including racemic mixtures, form part of this invention.

In addition, the compounds of the present invention can exist in unsolvated as well as solvated forms with pharmaceutically acceptable solvents such as water, ethanol, and the like. In general, the solvated forms are considered equivalent to the unsolvated forms for the purposes of the present invention.

The examples presented below are intended to illustrate particular embodiments of the invention, and are not intended to limit the scope of the specification or the claims in any way.

An illustration of the preparation of compounds of the present invention is shown in Schemes 1 to 3. R¹, R² and R³ are as defined above for Formula V and “Ar” represents aryl or substituted aryl. The definitions of R¹, R² and R³ correlate to the definitions of a R^{1′}, R^{2′} and R^{3′} in Formula I.

Armed with the disclosure provided herein (particularly the schemes and the synthetic examples that follow) and knowledge common to all who practice in the field, those of ordinary skill in the art will be able to make and use the entire scope of compounds disclosed herein.

The invention compounds can be prepared by any of several synthetic routes employing starting materials and intermediates readily available. The compounds of Formula I or V typically are prepared by coupling a 6-hydroxy-tetralone with a hydroxyalkyl-imidazole, as shown, for example, as the last step in Schemes 1 through 3. The reaction is carried out in the presence of a triarylphosphine and a dialkyl azodicarboxylate.

Scheme 1 shows a typical synthesis of the hydroxyalkyl-imidazole, starting from a readily available cyanoalkyl-imidazole. The secondary nitrogen of the imidazole is protected, for instance, with a t-BOC group, and the desired R³ group (e.g., arylalkyl such as benzyl) is then added by reacting the protected imidazole with R³-L, where L is a leaving group such as halo or hydroxy. The reaction generally is carried out in the presence of an acid such as sulfuric acid or methanesulfonic acid, which also removes the N-protecting group. The cyano group is next hydrolyzed to a carboxylic acid by reaction with a strong base such as sodium hydroxide, and the acid can be esterified by reaction with an alcohol. The ester is then readily reduced by reaction with lithium aluminum hydride or similar reducing agent to give the desired hydroxyalkyl-imidazole. The hydroxyalkyl-imidazole is then reacted with a suitably substituted 6-hydroxytetralone in the presence of an arylphosphine such as triphenylphosphine, and a dialkyl azodicarboxylate (e.g., a Mitsunobu esterification reaction).

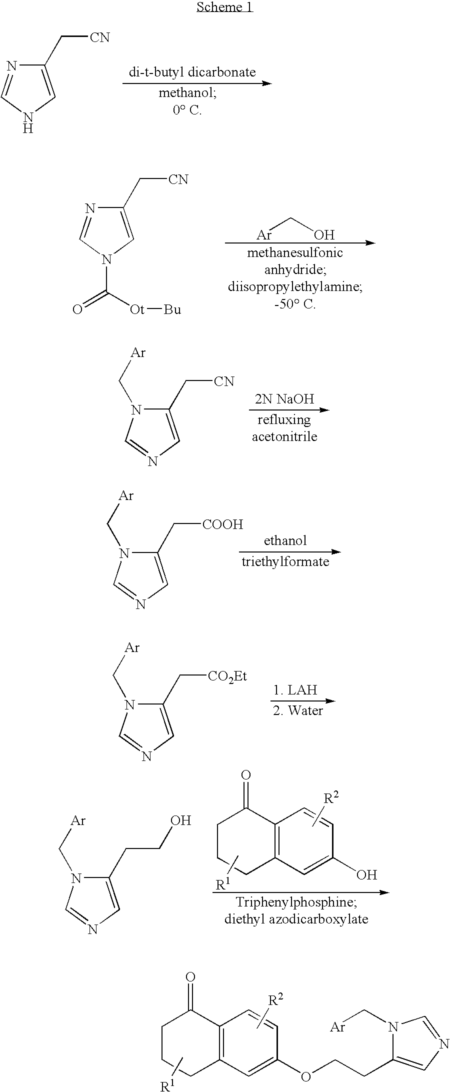

Scheme 2 shows another typical synthesis of an alkylimidazole derivative that can be coupled to a 6-hydroxy-tetralone to give invention compounds. In Scheme 2, a carboxyalkyl-imidazole is converted to an ester by reaction with an alcohol. The secondary amino nitrogen of the imidazole is protected with a common nitrogen protecting group such as triphenylmethyl. The protected imidazole is reacted with an arylalkyl halide (R³-L) to provide the desired substituted imidazole reactant, which is then coupled with the 6-hydroxy-tetralone as in Scheme 1.

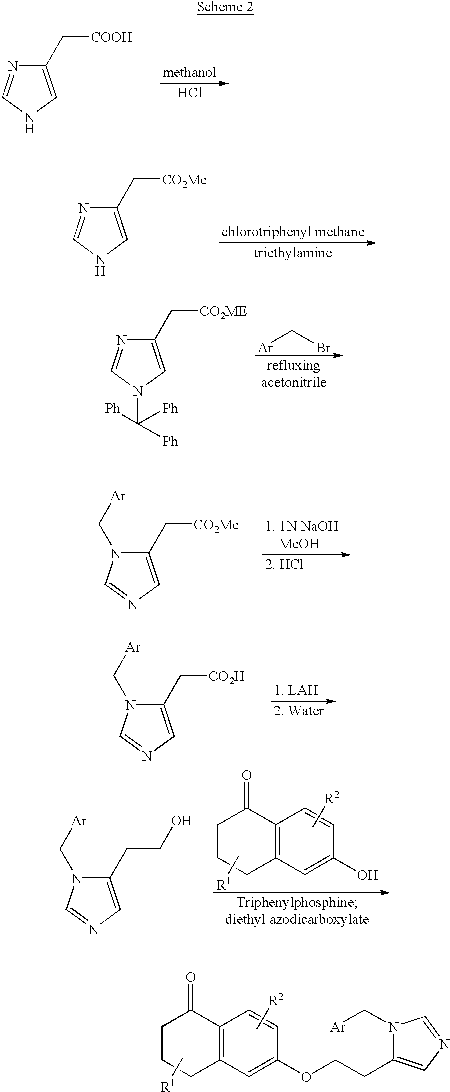

Scheme 3 illustrates another method for making substituted imidazoles which can be coupled to a 6-hydroxy-tetralone to provide invention compounds of Formulas I or V. The scheme starts with 4-formylimidazole. The τ (tau) nitrogen is protected with a typical nitrogen protecting group such as triphenylmethyl (trityl-TRT). The formyl group is converted to a secondary alcohol by reaction with an aryl lithium reagent (J. Heterocyclic Chemistry, 1993; 30(6), 1645-1651). The ζ (pi) ring nitrogen of the imidazole is derivatized by reaction with a suitable reagent, such as a aralkyl-protected alkoxy-L, where L is a leaving group, such as halogen, mesylate, or tosylate; preferably, a benzyl-protected alkoxyhalide. Removal of the protecting groups by standard methods affords a hydroxyalkyl-imidazole, which is coupled to the 6-hydroxy-tetralone as described in Scheme 1.

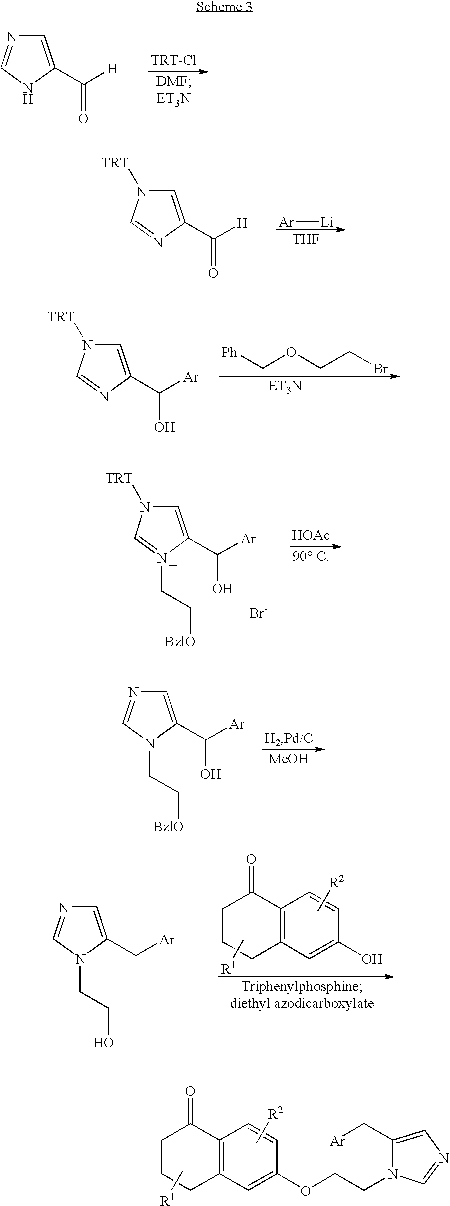

The disclosures in this application of all articles and references, including patents, are incorporated herein by reference.

The invention is illustrated further by the following examples which are not to be construed as limiting the invention in scope or spirit to the specific procedures described in them.

The starting materials and various intermediates may be obtained from commercial sources, prepared from commercially available organic compounds, or prepared using well known synthetic methods.

Representative examples of methods for preparing the compounds of the invention, as well as intermediates of the invention are set forth below.

### EXAMPLE 1

Synthesis of 6-{2-[3-(4-methoxy-3-methyl-benzyl)-3H-imidazol-4-yl]-ethoxy}-3,4-dihydro-2H-naphthalen-1-one (Compound 12)

1. 4-Cyanomethyl-imidazole-1-carboxylic Acid Tert-Butyl Ester

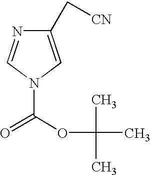

A solution of 4(5)-cyanomethylimidazole (12.2 g, 0.114 mol) in methanol (150 mL) is cooled to 0° C., and a solution of di-tert-butyl dicarbonate (30 g, 0.137 mol) in methanol (75 mL) is then added. The reaction is stirred under a nitrogen atmosphere and is left to warm to room temperature overnight. The solution is concentrated in vacuo and the residue taken up in isopropyl ether and concentrated in vacuo. The residue is taken up in isopropyl ether and chilled for several hours. The crystalline product is collected by filtration. The mother liquor is purified by flash chromatography (chloroform:acetone/9:1) to give 5.7 grams of the title compound (27% yield).

2. [3-(4-Methoxy-3-methyl-benzyl)-3H-imidazol-4-yl]-acetonitrile

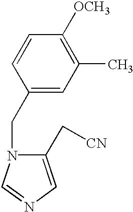

A solution of methanesulfonic anhydride (7.67 g, 0.044 mol) in methylene chloride is cooled to −50° C. Dropwise, a solution of 4-methoxy-3-methyl-benzyl alcohol (6.69 g, 0.044 mol), diisopropylethylamine (7.67 mL, 0.044 mol), and methylene chloride (60 mL) is added. The solution is stirred at −50° C. for 15 minutes and warmed to −20° C. over 15 minutes. The reaction vessel is cooled back to −50° C. and a solution of the product from step 1, 4-cyanomethyl-imidazole-1-carboxylic acid tert-butyl ester (9.07 g, 0.0438 mol), in methylene chloride (60 mL) is added dropwise. The reaction is warmed to room temperature and stirred overnight. A solution 0.25 M potassium phosphate buffer (pH 7, 300 mL) is added to the reaction mixture and vigorously stirred for 30 minutes. The organic phase is separated, washed with the phosphate buffer, dried over magnesium sulfate, and concentrated in vacuo. The product is purified by flash chromatography (0-1% methanol in chloroform) to give an oil which is dissolved in methylene chloride; the solution is evaporated and dried at 0.5 mm, to give a crystallized product (4. 3 g, 41% yield).

3. 3-(4-Methoxy-3-methyl-benzyl)-3H-imidazol-4-yl]-acetic Acid

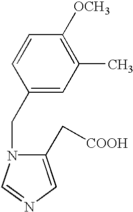

The product obtained in step 2, [3-(4-methoxy-3-methyl-benzyl)-3H-imidazol-4-yl]-acetonitrile (4.3 g, 0.0178 mol), is suspended in 2N NaOH (18 mL), and heated to reflux for 4 hours. The solution is cooled and neutralized with 1N HCl (36 mL), diluted with ethanol (100 mL) and concentrated in vacuo. The residue is taken up in ethanol (250 mL), the precipitate filtered and the solution filtered and concentrated in vacuo. The residue is triturated with hot ethyl acetate, cooled and filtered to give an off-white solid (4.5 g, 97% yield); mp: 117-121° C.

4. [3-(4-Methoxy-3-methyl-benzyl)-3H-imidazol-4-yl]-acetic Acid Ethyl Ester

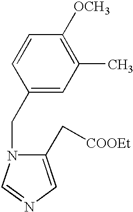

The product obtained in step 3, [3-(4-methoxy-3-methyl-benzyl)-3H-15 imidazol-4-yl]-acetic acid, (3.4 g, 0.013 mol), is dissolved in ethanol (100 mL) and triethylorthoformate (5 mL). This solution is saturated with dry HCl and the reaction is heated to reflux for 5 hours. The solution is concentrated in vacuo; the residue is triturated with ethyl acetate and dried overnight at 65° C., in vacuo to give 3.95 g (93% yield) of the desired product.

5. 2-[3-(4-Methoxy-3-methyl-benzyl)-3H-imidazol-4-yl]-ethanol

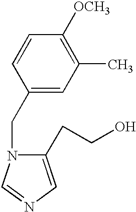

A suspension of the product from step 4, [3-(4-Methoxy-3-methyl-benzyl)-3H-imidazol-4-yl]-acetic acid ethyl ester (3.9 g, 0.012 mol), in tetrahydrofuran (250 mL) is stirred vigorously under a nitrogen atmosphere for 30 minutes. Lithium aluminium hydride (0.5 g, 0.013 mol) is then added slowly in 5 portions. The reaction is stirred 30 minutes at 0° C., warmed to room temperature for 3 hours, and then refluxed for 1 hour. The reaction is quenched by the dropwise addition of water (0.9 mL) and the Li/Al salts are removed by filtration through Celite, rinsing thoroughly with tetrahydrofuran:methanol (95:5). The filtrate is concentrated in vacuo to give the product (2.37 g, 80% yield).

6. 6-{2-[3-(4-Methoxy-3-methyl-benzyl)-3H-imidazol-4-yl]-ethoxy}-3,4-dihydro-2H-naphthalen-1-one

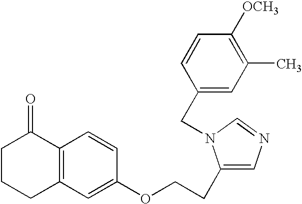

6-Hydroxy-tetralone (0.48 g, 0.003 mol) is dissolved in tetrahydrofuran (30 mL). Triphenylphosphine (1.11 g, 0.0042 mol) is then added followed by the product from step 5, 2-[3-(4-methoxy-3-methyl-benzyl)-3H-imidazol-4-yl]-ethanol (0.75 g, 0.003 mol). A solution of diethyl azodicarboxylate (0.6 mL, 0.0038 mol) in tetrahydrofuran (10 mL) is added slowly under a nitrogen atmosphere. The reaction is stirred at room temperature overnight under a nitrogen atmosphere. The precipitate is filtered, and the filtrate concentrated in vacuo. The residue is taken up in ethyl acetate, washed three times with brine, dried over magnesium sulfate, filtered, and concentrated in vacuo. The product is purified by flash chromatography (5% methanol in chloroform) to give 0.51 g (43% yield) material which is further purified by reverse phase HPLC (C-18 column; 22×250 mm; 0.1 mm; 300 Å; gradient: 10% to 40% acetonitrile; 0.1% trifluoroacetic acid; against 1% aqueous trifluoroacetic acid; 100 minutes; 13 mL/minute), to give 0.070 g of Compound 12 (6% yield).

MS: APCI: M+1: 391.2 (M: 390.5).

Analysis calculated for C₂₄H₂₆N₂O₃.1.25 CF₃COOH: C, 59.72; H, 5.15; N, 5.26. Found: C, 59.71; H, 5.13; N, 5.19.

### EXAMPLE 2

Synthesis of 6-[2-(3-benzyl-3H-imidazol-4-yl)-ethoxy]-5-propyl-3,4-dihydro-2H-naphthalen-1-one (Compound 2)

1. (1H-Imidazol-4-yl)-acetic Acid Ethyl Ester

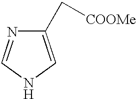

4-Imidazole acetic acid hydrochloride (5 g, 0.03 mol) is dissolved in methanol (100 mL) and the solution is saturated with dry HCl. The reaction is stirred overnight at room temperature under a nitrogen atmosphere. The solution is concentrated in vacuo and the residue dried to give the product (5.13 g, 0.029 mol).

2. (1-Trityl-1H-imidazol-4-yl)-acetic Acid Ethyl Ester

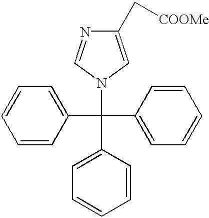

The product from step 1, (1H-imidazol-4-yl)-acetic acid ethyl ester, (5.13 g, 0.029 mol) is suspended in dimethylformamide (25 mL) and triethylamine (12.5 mL, 0.09 mol) is added followed by chlorotriphenyl methane (9.88 g, 0.036 mol). The suspension is stirred overnight at room temperature under a nitrogen atmosphere. Ethyl acetate (250 mL) is added to the reaction mixture followed by water (100 μL). The organic phase is collected and washed three times with saturated sodium bicarbonate, washed once with brine, dried over magnesium sulfate, filtered, and concentrated in vacuo to give the product (quantitative yield).

3. (3-Benzyl-3H-imidazol-4-yl)-acetic Acid Ethyl Ester

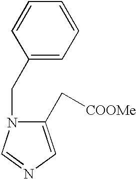

The product from step 2, (1-trityl-1H-imidazol-4-yl)-acetic acid ethyl ester (12.33 g, 0.032 mol), is dissolved in acetonitrile (100 mL). Benzylbromide (4.7 mL, 0.040 mol) is added and the reaction is refluxed overnight under a nitrogen atmosphere. The solution is cooled and concentrated in vacuo. The residue is dissolved in ethyl acetate and the resulting precipitate is filtered. The solid is dissolved in methanol and heated to reflux for 3 hours, and then stirred overnight at room temperature under a nitrogen atmosphere. The solution is concentrated and ethyl acetate is added to the residue. The precipitate obtained is filtered, washed with ethyl acetate, and dried. The solid is then suspended in 1:1 saturated sodium bicarbonate:methylene chloride and stirred for 2 hours. The organic phase is collected, dried over magnesium sulfate, filtered, concentrated and dried to give the product (3.80 g, 52% yield).

4. (3-Benzyl-3H-imidazol-4-yl)-acetic Acid

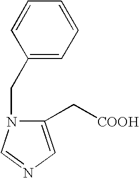

The product from step 3, (3-benzyl-3H-imidazol-4-yl)-acetic acid ethyl ester (3.80 g, 0.016 mol), is dissolved in methanol (50 mL) and tetrahydrofuran (50 mL), and 1N NaOH (48 mL) is then added. The reaction is stirred overnight at room temperature. 2N HCl (24 mL) is then added and the solution is concentrated in vacuo. The residue is suspended in water and lyophilized to give the product (6.28 g, quantitative yield).

5. 2-(3-Benzyl-3H-imidazol-4-yl)-ethanol

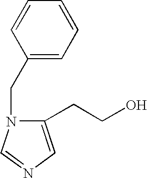

The product from step 4, (3-benzyl-3H-imidazol-4-yl)-acetic acid (6.28 g, 0.016 mol), is dissolved in tetrahydrofuran (50 mL). Lithium aluminium hydride (98%; 1.48 g, 0.038 mol) is suspended in tetrahydrofuran (50 mL) and the solution is added dropwise to the acid solution at room temperature under a nitrogen atmosphere. Once the addition is complete, the reaction is stirred for 1 hour at room temperature, 4 hours at reflux, and overnight at room temperature, all under a nitrogen atmosphere. The reaction is quenched by the addition of 1N sulfuric acid (1 mL) and diluted with ethyl acetate. The gray precipitate is filtered. The aqueous phase from the filtrate is concentrated and the residue dissolved in a minimal amount of water (100 mL). The pH is brought to 9 with the addition of 50% aqueous NaOH. The product is extracted with methylene chloride (5 times). The combined organic phases are washed twice with brine, dried over magnesium sulfate, filtered, and concentrated in vacuo to give the product (1.70 g, 52% yield).

6. 6-[2-(3-Benzyl-3I-imidazol-4-yl)-ethoxy]-5-propyl-3,4-dihydro-2H-naphthalen-1-one

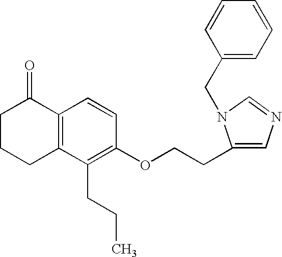

6-Hydroxy-5-propyl-3,4-dihydro-2H-naphthalen-1-one, prepared as described in Bioorganic & Medicinal Chemistry Letters, 1994;Vol. 4(24): 2883-2888, (0.43 g, 0.0021 mol) is dissolved in tetrahydrofuran (60 ML). Triphenylphosphine (0.86 g, 0.003 mol) is then added, followed by the product from step 5, 2-(3-benzyl-3H-imidazol-4-yl)-ethanol (0.51 g, 0.0025 mol). A solution of diethyl azodicarboxylate (0.5 mL, 0.0032 mol) in tetrahydrofuran (30 mL) is added slowly under a nitrogen atmosphere. The reaction is stirred at room temperature for 3 days under a nitrogen atmosphere. The solution is concentrated in vacuo. The residue is taken up in ethyl acetate, washed three times with brine, dried over magnesium sulfate, filtered, and concentrated in vacuo. The product is purified by flash chromoatography (0%-5% methanol in chloroform) and further purified by reverse phase HPLC (C-18 column; 22×250 mm; 0.1 mm; 300 Å; gradient: 10%-60% acetonitrile (0.1% trifluoroacetic acid) against 1% aqueous trifluoroacetic acid; 100 minutes; 13 mL/minute), to give 0.26 g of Compound 2 (32% yield).

MS: APCI: M+1: 389.3 (M: 388.5).

Analysis calculated for C₂₄H₂₆N₂O₃.1.88 CF₃COOH: C, 57.30; H, 5.00; N, 4.65. Found: C, 57.36; H, 4.93; N, 4.63.

### EXAMPLE 3

Synthesis of 6-[2-(3-benzyl-3H-imidazol-4-yl)-ethoxy]-5-phenethyl-3,4-dihydro-2H-naphthalen-1-one (Compound 3)

1. 6-Methoxy-5-phenylethynyl-3,4-dihydro-2H-naphthalen-1-one

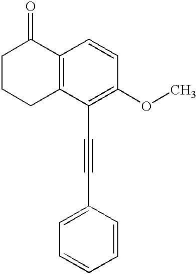

5-Bromo-6-methoxy-α-tetralone, prepared as described U.S. Pat. No. 4,618,683 Example 89, (20.41 g, 0.08 mol) is added to a mixture of dimethylformamide (160 mL) and triethylamine (80 mL) which has been purged with nitrogen gas to remove dissolved oxygen. This is followed by the addition of phenylacetylene (16.34 g, 0.16 mol), copper(I)iodide (0.5 g, 0.0026 mol), and dichlorobis(triphenylphosphine)palladium(II) (Aldrich Chemical Company; 2.24 g, 0.0026 mol). After stirring at 25° C. for 20 minutes, the mixture is heated to 108° C. for 2 hours. At that time, an additional amount of phenylacetylene is added (31 g, 0.303 mol) dropwise over 2 hours, followed by heating at 108° C. for 16 hours. The mixture is then evaporated in vacuo to remove solvent and excess reagent giving a syrup. The syrup is triturated with three fractions of hot ethyl ether (750 mL), which are combined and evaporated at 5° C. giving a suspended solid. The suspension is filtered and the solid is dried in vacuo giving 7.8 g of product. Additional fractions are recovered by hexane trituration of the ether insoluble residues, affording a total yield of product 10.3 g, 46.7%. This material is further purified by chromatography on 250 g silica gel eluted with a mixture of ethyl acetate/hexane (1:7). Appropriate fractions are evaporated to give a solid. mp 99° C. to 100° C., 7.5 g, 33.9% yield.

2. 6-Methoxy-5-phenethyl-3,4-dihydro-2H-naphthalen-1-one

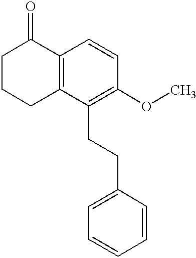

The product of step 1, 6-methoxy-5-phenylethynyl-3,4-dihydro-2H-naphthalen-1-one (5.5 g, 0.0199 mol), is dissolved in tetrahydrofuran (250 mL) to which is added 5% Pd/BaSO₄ catalyst Alfa #21162 unreduced, (1.4 g). The mixture is pressurized to 50 psi with H₂ gas and shaken at 25° C. for 1 hour. The mixture is filtered and evaporated to a solid, 5.7 g, 100% yield, mp 102° C. to 105° C. Structure is verified by NMR spectrum.

MS: APCI: M+1: 281.2 (M: 280.4).

3. 6-Hydroxy-5-phenethyl-3,4-dihydro-2H-naphthalen-1-one

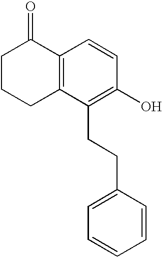

The product of step 2, 6-methoxy-5-phenethyl-3,4-dihydro-2H-naphthalen-1-one (5.5 g, 0.0196 g), is dissolved in dimethylsulfoxide (30 mL) and finely ground sodium cyanide (4.8 g, 0.096 mol) is added. The mixture is then placed alternately under vacuum and nitrogen atmosphere to remove dissolved oxygen and left under nitrogen atmosphere. The mixture is warmed to 180° C. for 5 hours, followed by cooling to 100° C. The solution is poured into 200 mL of rapidly stirred cold water and acidified by addition of concentrated HCl to pH 1. The solution is filtered and extracted three times with 200 mL ethyl ether. The ether extracts are dried over anhydrous magnesium sulfate, filtered, and evaporated to give a crystalline solid. The solid is filtered, washed with ethyl ether, and dried at 60° C. in vacuo giving a yellowish-green solid, 3.9 g, 75% yield. Structure is verified by NMR spectrum.

MS: APCI: M+1: 267.1 (M: 266.34).

4. 6-[2-(3-Benzyl-3H-imidazol-4-yl)-ethoxy]-5-phenethyl-3,4-dihydro-2H-naphthalen-1-one (Compound 3)

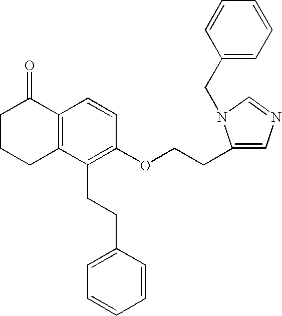

The product from step 3, 6-hydroxy-5-phenethyl-3,4-dihydro-2H-naphthalen-1-one (1.31 g, 0.005 mol), is dissolved in tetrahydrofuran (100 mL). Triphenyl phosphine (2.0 g, 0.0076 mol) is then added, followed by the product from step 5, Example 2, 2-(3-benzyl-3H-imidazol-4-yl)-ethanol (1.19 g, 0.0059 mol). A solution of diethyl azodicarboxylate (1.18 mL, 0.0075 mol) in tetrahydrofuran (30 mL) is added slowly under a nitrogen atmosphere. The reaction is stirred at room temperature for 5 days under a nitrogen atmosphere. The solution is concentrated in vacuo. The residue is taken up in ethyl acetate, washed three times with brine, dried over magnesium sulfate, filtered, and concentrated in vacuo. The product is purified by flash chromatography (0%-5% methanol in chloroform) and further purified by reverse phase HPLC (C-18 column; 22×250 mm; 0.1 mm; 300 Å; gradient: 10%-50% acetonitrile (0.1% trifluoroacetic acid) against 1% aqueous trifluoroacetic acid; 100 minutes; 13 mL/minute), to give 0.05 g of Compound 3 (2.2% yield).

MS: APCI: M+1: 451.2 (M: 450.6).

Analysis calculated for C₃₀H₃₀N₂O₂.1.06 CF₃COOH.0.75H₂O C, 65.95; H, 5.61; N, 4.79. Found: C, 65.99; H, 5.33; N, 4.61.

### EXAMPLE 4

Synthesis of 4-({5-[2-({5-oxo-1-[(2-pyridinylsulfonyl)methyl]-5,6,7,8-tetrahydro-2-naphthalenyl}oxy)ethyl]-1H-imidazol-1-yl}methyl)benzonitrile (Compound 28)

1. 4-{[5-(2-Hydroxyethyl)-1H-imidazol-1-yl]methyl}benzonitrile

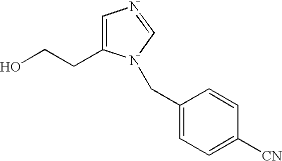

A mixture of 1-(triphenylmethyl)-4-(2-hydroxyethyl)imidazole (13 g, 36.7 mmol) [C. R. Ganellin et al., J. Med. Chem., 1996, 39, 3806] and 4-(bromo-methyl)benzonitrile (8.6 g, 44 mmol) in 100 mL of acetonitrile was heated under reflux for 2 days, and the solvent was removed under vacuum. The residue was then triturated twice with ethyl acetate, and the organic layer was discarded. The resulting residue was then dissolved in 100 mL of methanol and heated under reflux for 24 hours. The methanol was then removed under vacuum and the product was dissolved in 100 ml of 2M HCl. After being filtered to remove triphenylmethanol, the solution was made basic with NH₄OH, and the product was extracted into CH₂Cl₂ to give 2.24 g of a crude oil which was triturated with ethyl acetate to give a white solid, 1.05 g, 12.6% yield. mp 115-116° C. The NMR spectrum was consistent with structure. MS:APCI: M+1: 228.0 (M: 227.1).

2. 6-Hydroxy-5-(pyridin-2-ylsulfanylmethyl)-3,4-dihydro-2H-naphthalen-1-one

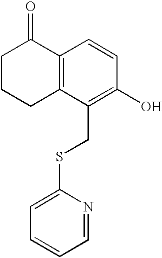

To tetrahydrofuran (500 ml) was added 2-mercaptopyridine (16 g, 0.14 mol) and triethylamine (20.8 ml, 0.15 mol). The mixture was warmed to 30° C., followed by the addition of a solution of 5-Chloromethyl-6-hydroxy-3,4-dihydro-2H-naphthalen-1-one, prepared as described in Chem. Pharm. Bull. 25(11) 2988-3002(1977), (29.5 g, 0.143 mol) in tetrahydrofuran 300 ml) giving a suspension. The mixture was stirred for 18 hours at 25° C. The suspension was filtered, and the filtrate was evaporated under vacuum giving a solid. The solid was dissolved in warm ethyl acetate, washed with 300 ml of water, brine, dried over anhydrous magnesium sulfate and filtered. The filtrate was concentrated in vacuo to a small volume, giving a suspended solid. The solid was filtered, washed with ethyl acetate, and dried in vacuo to a solid, 22.26 g, 56% yield. NMR spectrum was consistent with structure.

MS: APCI: M+1: 286.1 (M: 285.4).

3. 6-Hydroxy-5-(pyridine-2-sulfonylmethyl)-3,4-dihydro-2H-naphthalen-1-one

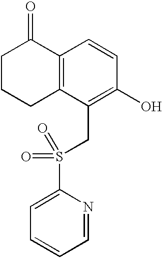

The product of step 2, 6-Hydroxy-5-(pyridin-2-ylsulfanylmethyl)-3,4-dihydro-2H-naphthalen-1-one (18 g, 0.063 mol), was added to dichloromethane (400 ml). A solution of m-chloroperbenzoic acid (70%, 33.6 g, 0.136 mol,) in dichloromethane (250 ml) was added over 2 hours, after which the mixture was allowed to stir for 18 hours at 25° C. The mixture was filtered, and the solid was resuspended in hot ethyl acetate and extracted with saturated sodium bicarbonate solution. The organic phase was washed with brine, dried over anhydrous magnesium sulfate and filtered. The filtrate was concentrated giving a solid, which was filtered, washed with ethyl acetate and dried, 6.8 g, 34% yield. NMR spectrum was consistent with structure.

MS: APCI: M+1: 318.3 (M: 317.4).

4. 4-({5-[2-({5-oxo-1-[(2-pyridinylsulfonyl)methyl]-5,6,7,8-tetrahydro-2-naphthalenyl}oxy)ethyl]-1H-imidazol-1-yl}methyl)benzonitrile

A mixture of the product of step 1, 4-{[5-(2-hydroxyethyl)-1H-imidazol-1-yl]methyl}benzonitrile (0.26 g, 1.14 mmol), the product of 3, 6-hydroxy-5-(pyridine-2-sulfonylmethyl)-3,4-dihydro-2H-naphthalen-1-one (0.32 g, 1 mmol) and triphenylphosphine (0.42 g, 1.6 mmol) in 5 mL dry tetrahydrofuran was cooled to 0° C. and treated dropwise with diisopropyl azodicarboxylate (0.3 g, 1.5 mmol). The resulting mixture was stirred at room temperature for 2 days and the solvent was removed. The residue was extracted into dichloromethane, washed with water, and dried over Na₂SO₄. After evaporation of the solvent, the residue was chromatographed on silica gel, eluting with 5% methanol in dichloromethane, to give 0.28 g (53% yield) of crude product, a sample of which was further purified by high pressure liquid chromatography [Bondelone-10 C18 column eluting with a mixture of 58% acetonitrile-water (9:1) and 42% 0.045 M NH₄HCO₂ (pH 3.49)] to give Compound 28 as a white solid (99% pure by HPLC). FABHRMS 527.17713 (MH⁺517.17530).

### EXAMPLE 5

Synthesis of 4-({5-[2-({1-[(isopropylsulfonyl)methyl]-5-oxo-5,6,7,8-tetrahydro-2-naphthalenyl}oxy)ethyl]-1H-imidazol-1-yl}methyl)benzonitrile (Compound 29)

1. 6-Hydroxy-5-isopropylsulfanylmethyl-3,4-dihydro-2H-naphthalen-1-one

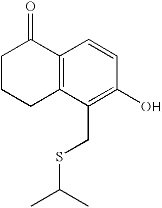

Tetrahydrofuran (100 ml, anhydrous, distilled) was sparged with nitrogen gas followed by addition of triethylamine (6.97 ml, 50 mmol) and 2-propanethiol (7 ml, 150 mmol). A solution of 5-Chloromethyl-6-hydroxy-3,4-dihydro-2H-naphthalen-1-one, prepared as described in Chem. Pharm. Bull. 25(11) 2988-3002(1977), (10.53 g, 50 mmol) in tetrahydrofuran (175 ml) was added. The mixture was stirred for 1 hour at 25° C., followed by heating at 85° C. for 3 hours. The mixture was filtered, and the filtrate was evaporated under vacuum giving a solid. The solid was dissolved in ethyl acetate, washed with IN citric acid and brine. The organic phase was separated, dried over anhydrous magnesium sulfate and filtered. The filtrate was concentrated in vacuo giving a solid which was filtered, washed with ethyl ether, and dried in vacuo to a solid, 8.69 g, 69.4% yield. NMR spectrum was consistent with structure.

MS: APCI: M+1: 251.2 (M: 250.4).

2. 6-Hydroxy-5-isopropylsulfonylmethyl-3,4-dihydro-2H-napthalen-1-one

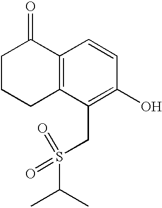

To dichloromethane (100 ml) was added the product of step 1, 6-Hydroxy-5-isopropylsulfanylmethyl-3,4-dihydro-2H-naphthalen-1-one (3.74 g, 14.9 mmol). The mixture was cooled to 15° C., followed by the addition of m-chloroperbenzoic acid (70% with water, 7.36 g, 29.9 mmol) over 2 minutes. The mixture stirred for hours at 25° C. giving a suspended solid. The suspension was evaporated under vacuum giving a solid. The solid was dissolved in warm ethyl acetate (700 ml), washed with saturated sodium bicarbonate and brine. The organic phase was separated, dried over anhydrous magnesium sulfate and filtered. The filtrate was concentrated in vacuo to 150 ml in volume at 2° C., giving a suspended solid. The solid was filtered, washed with ethyl ether, and dried in vacuo to a solid, 2.64 g, 63% yield. NMR spectrum was consistent with structure.

MS: APCI: M+1: 283.0 (M: 282.36).

3. A mixture of product of Example 4, step 1, 4-{[5-(2-hydroxyethyl)-1H-imidazol-1-yl]methyl}benzonitrile (0.26 g, 1.14 mmol), the product of 2, 6-hydroxy-5-isopropylsulfonylmethyl-3,4-dihydro-2H-naphthalen-1-one (0.28 g, 1 mmol), and triphenylphosphine (0.42 g, 1.6 mmol) in 5 mL dry tetrahydrofuran was cooled to 0° C. and treated dropwise with diisopropyl azodicarboxylate (0.3 g, 1.5 mmol). The resulting mixture was stirred at room temperature for 2 days and the solvent was removed. The residue was extracted into dichloromethane, washed with water, and dried over Na₂SO₄. After evaporation of the solvent, the residue was chromatographed on silica gel, eluting with 5% methanol in dichloromethane, to give a crude product which was further purified by high pressure liquid chromatography [Bondclone-10 C18 column eluting with a mixture of 58% acetonitrile-water (9:1) and 42% 0.045 M NH₄HCO₂ (pH 3.49)] to give Compound 29 as a white solid, 0.06 g (12% yield, 95% pure by HPLC). FABHRMS 492.19678 (MH+492.19570).

### EXAMPLE 6

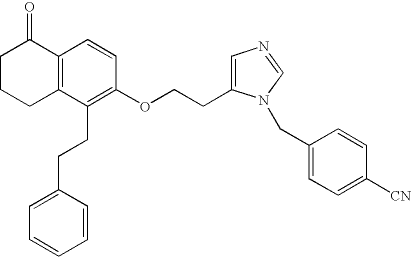

Synthesis of 4-{[5-(2-{[5-oxo-1-(2-phenylethyl)-5,6,7,8-tetrahydro-2-naphthalenyl]oxy}ethyl)-1H-imidazol-1-yl]methyl}benzonitrile (Compound 4)

1. A mixture of the product of Example 4, step 1, 4-{[5-(2-hydroxyethyl)-1H-imidazol-1-yl]methyl}benzonitrile (1.34 g, 5 mmol), the product of Example 3, step 3, 6-hydroxy-5-(2-phenylethyl)-3,4-dihydro-1(2H)-naphthalenone (1.14 g, 5 mmol) and triphenylphosphine (1.97 g, 7.5 mmol) in 25 mL dry tetrahydrofuran was cooled to 0° C. and treated dropwise with diisopropyl azodicarboxylate (1.30 g, 7.5 mmol). The resulting mixture was stirred at room temperature for 5 days and the solvent was removed. The residue was extracted into dichloromethane, washed with water, and dried over Na₂SO₄. After evaporation of the solvent, the residue was chromatographed on silica gel, eluting with ethyl acetate-methanol 3:1, to give a crude product which was further purified by chromatography on alumina, eluting with dichloromethane-methanol 99:1, to give a white solid, 1.29 g (54% yield). Recrystallization from dichloromethane-isopropanol gave material melting at 179-180.5° C.

Analysis calculated for C₃₁H₂₉N₃O₂: Theory: C, 78.29; H, 6.15; N, 8.84. Found: C, 78.12; H, 6.28; N, 8.89.

### EXAMPLE 7

The following compounds are prepared essentially according to the procedures described in Examples 1 to 6 and shown in Schemes 1 to 3:

(a) 4-{5-[2-(5-oxo-5,6,7,8-tetrahydronaphthalen-2-yloxy)ethyl]imidazol-1-ylmethyl}benzonitrile (Compound 1);

(b) 4-(2-{2-[2-(3-benzyl-3H-imidazol-4-yl)ethoxy]-5-oxo-5,6,7,8-tetrahydronaphthalen-1-yl}ethyl)benzoic acid (Compound 5);

(c) 6-[2-(3-benzyl-3H-imidazol-4-yl)ethoxy]-5-phenylaminomethyl-3,4-dihydro-2H-naphthalene-1-one (Compound 6);

(d) 5-benzyl-6-[2-(3-benzyl-3H-imidazol-4-yl)ethoxy]-3,4-dihydro-2H-naphthalene-1-one (Compound 7);

(e) 6-[2-(3-benzyl-3H-imidazol-4-yl)ethoxy]-5-phenylamino-3,4-dihydro-2H-naphthalene-1-one (Compound 8);

(f) N-{2-[2-(3-benzyl-3H-imidazol-4-yl)ethoxy]-5-oxo-5,6,7,8-tetrahydro-naphthalene-1-yl}benzamide (Compound 9);

(g) 6-[2-(3-benzyl-3H-imidazol-4-yl)ethoxy]-5-isopropoxymethyl-3,4-dihydro-2H-naphthalene-1-one (Compound 10);

(h) 3-{2-[2-(3-benzyl-3H-imidazol-4-yl)ethoxy]-5-oxo-5,6,7,8-tetrahydro-naphthalene-1-yl}propionic acid methyl ester (Compound 11);

(i) 6-[2-(3-benzyl-3H-imidazol-4-yl)-1-phenylethoxy]-3,4-dihydro-2H-naphthalene-1-one (Compound 13);

(j) 4-{3-[2-(5-oxo-5,6,7,8-tetrahydronaphthalen-2-yloxy)ethyl]-3H-imidazol-4-yl}methyl)benzonitrile (Compound 14);

(k) 6-[2-(5-benzyl-imidazol-1-yl)ethoxy]-5-propyl-3,4-dihydro-2H-naphthalene-1-one (Compound 15);

(l) 6-[2-(5-benzyl-imidazol-1-yl)ethoxy]-5-phenethyl-3,4-dihydro-2H-naphthalene-1-one (Compound 16);

(m) 4-{3-[2-(5-oxo-1-phenethyl-5,6,7,8-tetrahydronaphthalen-2-yloxy)ethyl]-3H-imidazol-4-yl}methyl)benzonitrile (Compound 17);

(n) 4-(2-{2-[2-(5-benzyl-imidazol-1-yl)ethoxy]-5-oxo-5,6,7,8-tetrahydronaphthalen-1-yl} ethyl)benzoic acid (Compound 18);

(o) 6-[2-(5-benzyl-imidazol-1-yl)ethoxy]-5-phenylaminomethyl-3,4-dihydro-2H-naphthalene-1-one (Compound 19);

(p) 5-benzyl-6-[2-(5-benzyl-imidazol-1-yl)ethoxy]-3,4-dihydro-2H-naphthalene-1-one (Compound 20);

(q) 6-[2-(5-benzyl-imidazol-1-yl)ethoxy]-5-phenylamino-3,4-dihydro-2H-naphthalene-1-one (Compound 21);

(r) N-{2-[2-(5-benzyl-imidazol-1-yl)ethoxy]-5-oxo-5,6,7,8-tetrahydro-naphthalene-1-yl}benzamide (Compound 22);

(s) 6-{2-[3-(methoxy-3-methylbenzyl)-3H-imidazol-4-yl]ethoxy}-5-phenethyl-3,4-dihydro-2H-naphthalene-1-one (Compound 23);

(t) 6-[2-(5-Benzyl-3H-imidazol-1-yl)-ethoxy]-5-(2-pyridin-2-yl-ethyl)-3,4-dihydro-2H-naphthalene-1-one (Compound 24);

(u) 6-{2-[3-(4-Methoxy-3-methylbenzyl)-3H-imidazol-4-yl]ethoxy}-5-(2-pyridin-2-ylethyl)-3,4-dihydro-2H-naphthalene-1-one (Compound 25);

(v) 5-Benzenesulfonylmethyl-6-{2-[5-(4-methoxy-3-methylbenzyl)imidazol-1-yl]ethoxy}-3,4-dihydro-2H-naphthalene-1-one (Compound 26); and

(w) 5-Benzenesulfonylmethyl-6-{2-[3-(4-methoxy-3-methylbenzyl)-3H-imidazol-4-yl]ethoxy}1-3,4-dihydro-2H-naphthalene-1-one (Compound 27).

### EXAMPLE 8

The pharmaceutical utility of compounds of this invention was established by the following standard assay which measures inhibition of the enzyme protein:farnesyl transferase (PFT), also referred to as farnesyl protein transferase (FPT).

PFT Inhibitory Activity

The PFT (or FPT) inhibitory activity of compounds of the present invention was assayed in HEPES buffer (pH 7.4) containing 5 mM potassium phosphate and 20 μM ZnCl₂. The solution also contained 5 mM DTT (dithiothreitol), 5 mM MgCl₂, and 0.1% PEG 8000. Assays were performed in 96 well plates (Wallec) and employed solutions composed of varying concentrations of a compound of the present invention in 10% DMSO (dimethylsulfoxide). Upon addition of both substrates, radiolabeled farnesyl pyrophosphate ([1³H], specific activity 15-30 Ci/mmol, final concentration 134 nM) and (biotinyl)-Ahe-Thr-Lys-Cys-Val-Ile-Met ([3aS[3a alpha, 4 beta, 6a alpha]-hexahydro-2-oxo-1H-thieno[3,4-d]imidazole-5-pentanoic acid]-[7-aminoheptanoic acid]-Thr-Lys-Cys-Val-Ile-Met) (Ahe is 7-aminoheptanoic acid, Thr is threonine, Lys is lysine, Cys is cysteine, Val is valine, Ile is isoleucine, and Met is methionine) (final concentration 0.2 μM), the enzyme reaction was started by addition of SF9 affinity purified rat FPT. After incubation at 30° C. for 30 minutes, the reaction was terminated by diluting the mixture 2.5-fold with a stop buffer containing 1.5 M magnesium acetate, 0.2 M H₃PO_{4, 0.5}% BSA (bovine serum albumin), and strepavidin beads (Amersham) at a concentration of 1.3 mg/mL. After allowing the plate to settle for 30 minutes at room temperature, radioactivity was quantitated on a microBeta counter (Model 1450, Wallec). The assay was also carried out without 5 mM potassium phosphate. The IC₅₀ values (the micromolar amount of invention compound required to inhibit enzyme activity by 50%) of compounds of Formula I or V range from about 0.01 to about 50.0. The inhibitory activity of specific representative compounds are shown in Table 2.

TABLE 2

| | IC₅₀ (μm) | IC₅₀ |
|---|---|---|
| | Hepes ± 5 mM | (μm) |
| Compound No. | Phosphate | Hepes |
| 2 | 0.87 | 1.39 |
| 3 | 0.052 | 0.073 |
| 4 | — | 0.0003 |
| 12 | 0.022 | 0.024 |
| 28 | — | 0.0003 |
| 29 | — | 0.0005 |

The enzyme inhibitory activity of the invention compounds, as established in the foregoing assay, demonstrates that the compounds are useful in preventing and treating uncontrolled cellular proliferation, and are thus useful for preventing and treating disease states characterized by such proliferation. The compounds of Formula I or V will be used in the form of pharmaceutical formulations, and the following examples illustrate typical dosage forms.

### EXAMPLE 9

Tablet Formulation

| Ingredient | Amount |
|---|---|
| Compound No. 12 | 50 mg |
| Lactose | 80 mg |
| Cornstarch (for mix) | 10 mg |
| Cornstarch (for paste) | 8 mg |
| Magnesium Stearate (1%) | 2 mg |
| | 150 mg |

Compound No. 12 is mixed with the lactose and cornstarch (for mix) and blended to uniformity to a powder. The cornstarch (for paste) is suspended in 6 mL of water and heated with stirring to form a paste. The paste is added to the mixed powder, and the mixture is granulated. The wet granules are passed through a No. 8 hard screen and dried at 50° C. The mixture is lubricated with 1% magnesium stearate and compressed into a tablet. The tablets are administered to a patient at the rate of 1 to 4 each day for prevention and treatment of atherosclerosis or cancer.

### EXAMPLE 10

### Parenteral Solution

In a solution of 700 mL of propylene glycol and 200 mL of water for injection is added 20.0 g of Compound No. 8. The mixture is stirred and the pH is adjusted to 5.5 with hydrochloric acid. The volume is adjusted to 1000 mL with water for injection. The solution is sterilized, filled into 5.0 mL ampoules, each containing 2.0 mL (40 mg of Compound No. 8), and sealed under nitrogen. The solution is administered by injection to a patient suffering from cancer and in need of treatment.

### EXAMPLE 11

### Patch Formulation

Compound No. 26 (10 mg) is suspended in a mixture of mineral oil, polyisobutylene, and colloidal silicon dioxide (5 mg each). This mixture is applied evenly to a 10 cm² microporous polypropylene membrane (which has a backing layer of pigmented polyester film) that controls the rate of delivery of active agent to the skin surface of a patient. The membrane is layered onto an adhesive formulation of polyisobutylene, and the mixture is covered with a protective slit release liner of polyester that is removed immediately before applying the patch to the chest or forearm of a patient to treat for example, Alzheimer's disease or cancer.

The invention and the manner and process of making and using it, are now described in such full, clear, concise, and exact terms as to enable any person skilled in the art to which it pertains, to make and use the same. It is to be understood that the foregoing describes preferred embodiments of the present invention and that modifications may be made therein without departing from the spirit or scope of the present invention as set forth in the claims. To particularly point out and distinctly claim the subject matter regarded as invention, the following claims conclude this specification.

What is claimed is:

1. A compound of formula 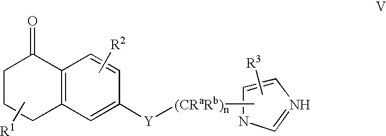
and pharmaceutically acceptable salts, esters, amides, and prodrugs thereof,
wherein:
R^{a}, R^{b}, and R^{c} are independently hydrogen, (C₁-C₆)-alkyl, (C₂-C₆)-alkenyl, aryl, heteroaryl, arylalkyl, or heteroarylalkyl, wherein the aryl, heteroaryl, arylalkyl, or heteroarylalkyl is optionally substituted with one, two, or three groups independently selected from the group consisting of alkyl, O-alkyl, S-alkyl, OH, SH, —CN, halogen, 1,3-dioxolanyl, CF₃, NO₂, NH₂, NHCH₃, N(CH₃)₂, NHCO-alkyl, —(CH₂)ₘCO₂H, —(CH₂)ₘCO₂-alkyl, —(CH₂)ₘSO₃H, —NH alkyl, —N(alkyl)₂, —(CH₂)ₘPO₃H₂, —(CH₂)ₘPO₃(alkyl)₂, —(CH₂)ₘSO₂NH₂, and —(CH₂)ₘSO₂NH-alkyl, wherein m is 0, 1, 2, or 3;
R¹ and R² are independently hydrogen, (C₁-C₆)-alkyl, (C₂-C₆)-alkenyl, aryl, heteroaryl, arylalkyl, or heteroarylalkyl, wherein the aryl, heteroaryl, arylalkyl or heteroarylalkyl is optionally substituted with one, two, or three groups independently selected from the group consisting of alkyl, O-alkyl, S-alkyl, OH, SH, —CN, halogen, 1,3-dioxolanyl, CF₃, NO₂, NH₂, NHCH₃, N(CH₃)₂, NHCO-alkyl, —(CH₂)ₘCO₂H, —(CH₂)ₘCO₂-alkyl, —(CH₂)ₘSO₃H, —NH-alkyl, —N(alkyl)₂, —(CH₂)ₘPO₃H₂, —(CH₂)ₘPO₃ (alkyl)₂, —(CH₂)ₘSO₂NH₂, —(CH₂)ₘ-heteroaryl, —(CH₂)ₘS-aryl, —(CH₂)ₘS-heteroaryl, —(CH₂)ₘSO₂-aryl, —(CH₂)ₘSO₂-heteroaryl, and —(CH₂)ₘSO₂NH-alkyl, wherein m is 0, 1, 2, or 3, and wherein each of the R¹ and R² groups can be attached through a linker, or through a lower alkyl optionally interrupted by a linker, said linker selected from the group consisting of 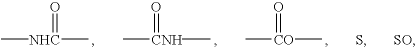
SO₂, O, and NR^{c};
Y is NR^{c}, O, —CHR^{c}, or S;
n is 0, 2, or 3, provided that when the imidazole is attached at the imidazole nitrogen to (CR^{a}R^{b})ₙ and Y is O, NR^{c} or S, then n is not 0; and
R³ is aryl, heteroarylalkyl, or arylalkyl, wherein the aryl, heteroaryl or arylalkyl is optionally substituted with up to three groups selected from the group consisting of halogen, (C₁-C₆)-alkyl, amino, (C₁-C₆)-alkoxy, hydroxy, trifluoromethyl, mono- or dialkylamino, (C₁-C₆)-thioalkoxy, cyano, nitro, 1,3-dioxolanyl, NHCO(C₁-C₆)-alkyl, (CH₂)ₘCO₂H, (CH₂)ₘCO₂(C₁-C₆)-alkyl, (CH₂)ₘSO₃H, —(CH₂)ₘPO₃H₂, (CH₂)ₘPO₃ [(C₁-C₆)-alkyl]2, (CH₂)ₘSO₂NH₂, and (CH₂)ₘSO₂NH(C₁-C₆)-alkyl, wherein m is 0, 1, 2, or 3.

2. A compound of formula 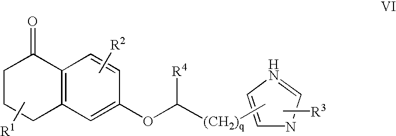
and pharmaceutically acceptable salts, esters, amides, and prodrugs thereof,
wherein:
R¹ and R² are independently hydrogen, (C₁-C₆)-alkyl, (C₂-C₆)-alkenyl, aryl, heteroaryl, arylalkyl, or heteroarylalkyl, wherein the aryl, heteroaryl, arylalkyl or heteroarylalkyl is optionally substituted with one, two, or three groups independently selected from the group consisting of alkyl, O-alkyl, S-alkyl, OH, SH, —CN, halogen, 1,3-dioxolanyl, CF₃, NO₂, NH₂, NHCH₃, N(CH₃)₂, NHCO-alkyl, —(CH₂)ₘCO₂H, —(CH₂)ₘCO₂-alkyl, —(CH₂)ₘSO₃H, —NH-alkyl, —N(alkyl)₂, —(CH₂)ₘPO₃H₂, —(CH₂)ₘPO₃(alkyl)₂, —(CH₂)ₘSO₂NH₂, (CH₂)ₘ-heteroaryl, —(CH₂)ₘS-aryl, —(CH₂)ₘS-heteroaryl, —(CH₂)ₘSO₂-aryl, —(CH₂)ₘSO₂-heteroaryl, and —(CH₂)ₘSO₂NH-alkyl, wherein m is 0, 1, 2, or 3; and wherein each of the R¹ and R² groups can be attached through a linker, or through a lower alkyl optionally interrupted by a linker, said linker selected from the group consisting of 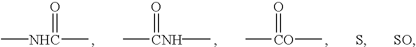
SO₂, O, and NR^{c};
R^{c} is hydrogen, (C₁-C₆)-alkyl, or aryl;
q is 1 or 2;
R⁴ is hydrogen, heteroaryl, or aryl, wherein the aryl or heteroaryl is optionally substituted with up to three groups selected from the group consisting of halogen, (C₁-C₆)-alkyl, amino, (C₁-C₆)-alkoxy, hydroxy, trifluoromethyl, mono- or dialkylamino, (C₁-C₆)-thioalkoxy, cyano, nitro, 1,3-dioxolanyl, NHCO(C₁-C₆)-alkyl, (CH₂)ₘCO₂H, (CH₂)ₘCO₂(C₁-C₆)-alkyl, (CH₂)ₘSO₃H, —(CH₂)ₘPO₃H₂, (CH₂)ₘPO₃ [(C₁-C₆)-alkyl]₂, (CH₂)ₘSO₂NH₂, and (CH₂)ₘSO₂NH(C₁-C₆)-alkyl, wherein m is 0, 1, 2, or 3; and
R³ is aryl, heteroarylalkyl, or arylalkyl, wherein the aryl, heteroaryl or arylalkyl is optionally substituted with up to three groups selected from the group consisting of halogen, (C₁-C₆)-alkyl, amino, (C₁-C₆)-alkoxy, hydroxy, trifluoromethyl, mono- or dialkylamino, (C₁-C₆)-thioalkoxy, cyano, nitro, 1,3-dioxolanyl, NHCO(C₁-C₆)-alkyl, (CH₂)ₘCO₂H, (CH₂)ₘCO₂(C₁-C₆)-alkyl, (CH₂)ₘSO₃H, (CH₂)ₘPO₃H₂, (CH₂)ₘPO₃ [(C₁-C₆)-alkyl]2, (CH₂)ₘSO₂NH₂, and (CH₂)ₘSO₂NH(C₁-C₆)-alkyl, wherein m is 0, 1, 2, or 3.

3. A compound of formula 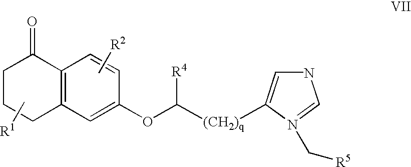
and pharmaceutically acceptable salts, esters, amides, and prodrugs thereof,
wherein:
R¹ and R² are independently hydrogen, (C₁-C₆)-alkyl, (C₂-C₆)-alkenyl, aryl, heteroaryl, arylalkyl, or heteroarylalkyl, wherein the aryl, heteroaryl, arylalkyl or heteroarylalkyl is optionally substituted with one, two, or three groups independently selected from the group consisting of alkyl, O-alkyl, S-alkyl, OH, SH, —CN, halogen, 1,3-dioxolanyl, CF₃, NO₂, NH₂, NHCH₃, N(CH₃)₂, NHCO-alkyl, —(CH₂)ₘCO₂H, —(CH₂)ₘCO₂-alkyl, —(CH₂)ₘSO₃H, —NH-alkyl, —N(alkyl)₂, —(CH₂)ₘPO₃H₂, —(CH₂)ₘPO₃ (alkyl)₂, —(CH₂)ₘSO₂NH₂, —(CH₂)ₘ-heteroaryl, —(CH₂)ₘS-aryl, —(CH₂)ₘS-heteroaryl, —(CH₂)ₘSO₂-aryl, —(CH₂)ₘSO₂-heteroaryl, and —(CH₂)ₘSO₂NH-alkyl, wherein m is 0, 1, 2, or 3; and wherein each of the R¹ and R² groups can be attached through a linker, or through a lower alkyl optionally interrupted by a linker, said linker selected from the group consisting of 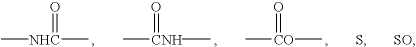
SO₂, O, and NR^{c};
R^{c} is hydrogen, (C₁-C₆)-alkyl, or aryl;
q is 1 or 2;
R⁴ is hydrogen, heteroaryl, or aryl, wherein the aryl or heteroaryl is optionally substituted with up to three groups selected from the group consisting of halogen, (C₁-C₆)-alkyl, amino, (C₁-C₆)-alkoxy, hydroxy, trifluoromethyl, mono- or dialkylamino, (C₁-C₆)-thioalkoxy, cyano, nitro, 1,3-dioxolanyl, NHCO(C₁-C₆)-alkyl, (CH₂)ₘCO₂H, (CH₂)ₘCO₂(C₁-C₆)-alkyl, (CH₂)ₘSO₃H, —(CH₂)ₘPO₃H₂, (CH₂)ₘPO₃ [(C₁-C₆)-alkyl]₂, (CH₂)ₘSO₂NH₂, and (CH₂)ₘSO₂NH(C₁-C₆)-alkyl, wherein m is 0, 1, 2, or 3; and
R⁵ is aryl optionally substituted with up to three groups selected from the group consisting of halogen, (C₁-C₆)-alkyl, amino, (C₁-C₆)-alkoxy, hydroxy, trifluoromethyl, mono- or dialkylamino, (C₁-C₆)-thioalkoxy, cyano, nitro, 1,3-dioxolanyl, NHCO(C₁-C₆)-alkyl, (CH₂)ₘCO₂H, (CH₂)ₘCO₂(C₁-C₆)-alkyl, (CH₂)ₘSO₃H, (CH₂)ₘPO₃H₂, (CH₂)ₘPO₃ [(C₁-C₆)-alkyl]₂, (CH₂)ₘSO₂NH₂, and (CH₂)ₘSO₂NH(C₁-C₆)-alkyl, wherein m is 0, 1, 2, or 3.

4. A compound of formula 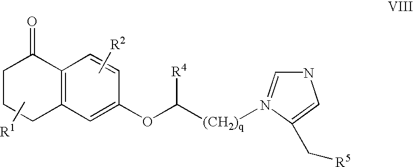
and pharmaceutically acceptable salts, esters, amides, and prodrugs thereof,
wherein:
R¹ and R² are independently hydrogen, (C₁-C₆)-alkyl, (C₂-C₆)-alkenyl, aryl, heteroaryl, arylalkyl, or heteroarylalkyl, wherein the aryl, heteroaryl, arylalkyl or heteroarylalkyl is optionally substituted with one, two, or three groups independently selected from the group consisting of alkyl, O-alkyl, S-alkyl, OH, SH, —CN, halogen, 1,3-dioxolanyl, CF₃, NO₂, NH₂, NHCH₃, N(CH₃)₂, NHCO-alkyl, —(CH₂)ₘCO₂H, —(CH₂)ₘCO₂-alkyl, —(CH₂)ₘSO₃H, —NH-alkyl, —N(alkyl)₂, —(CH₂)ₘPO₃H₂, —(CH₂)ₘPO₃ (alkyl)₂, —(CH₂)ₘSO₂NH₂, —(CH₂)ₘ-heteroaryl, —(CH₂)ₘS-aryl, —(CH₂)ₘS-heteroaryl, —(CH₂)ₘSO₂-aryl, —(CH₂)ₘSO₂-heteroaryl, and —(CH₂)ₘSO₂NH-alkyl, wherein in is 0, 1, 2, or 3; and wherein each of the R¹ and R² groups can be attached through a linker, or through a lower alkyl optionally interrupted by a linker, said linker selected from the group consisting of 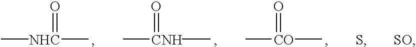
SO₂, O, and NR^{c};
R^{c} is hydrogen, (C₁-C₆)-alkyl, or aryl;
q is 1 or 2;
R⁴ is hydrogen, heteroaryl, or aryl, wherein the aryl or heteroaryl is optionally substituted with up to three groups selected from the group consisting of halogen, (C₁-C₆)-alkyl, amino, (C₁-C₆)-alkoxy, hydroxy, trifluoromethyl, mono- or dialkylamino, (C₁-C₆)-thioalkoxy, cyano, nitro, 1,3-dioxolanyl, NHCO(C₁-C₆)-alkyl, (CH₂)ₘCO₂H, (CH₂)ₘCO₂(C₁-C₆)-alkyl, (CH₂)ₘSO₃H, —(CH₂)ₘPO₃H₂, (CH₂)ₘPO₃ [(C₁-C₆)-alkyl]2, (CH₂)ₘSO₂NH₂, and (CH₂)ₘSO₂NH(C₁-C₆)-alkyl, wherein m is 0, 1, 2, or 3; and
R⁵ is aryl optionally substituted with up to three groups selected from the group consisting of halogen, (C₁-C₆)-alkyl, amino, (C₁-C₆)-alkoxy, hydroxy, trifluoromethyl, mono- or dialkylamino, (C₁-C₆)-thioalkoxy, cyano, nitro, 1,3-dioxolanyl, NHCO(C₁-C₆)-alkyl, (CH₂)ₘCO₂H, (CH₂)ₘCO₂(C₁-C₆)-alkyl, (CH₂)ₘSO₃H, —(CH₂)ₘPO₃H₂, (CH₂)ₘPO₃ [(C₁-C₆)-alkyl]₂, (CH₂)ₘSO₂NH₂, and (CH₂)ₘSO₂NH(C₁-C₆)-alkyl, wherein m is 0, 1, 2, or 3.

5. A compound according to claim 1 wherein R¹ is hydrogen.

6. A compound according to claim 1 wherein R² is hydrogen, lower alkyl, arylalkyl, arylaminoalkyl, arylamino, arylcarbonylamino, alkoxyalkyl, or alkoxycarbonylalkyl.

7. A compound according to claim 1 wherein Y is O.

8. A compound according to claim 1 wherein n is 2.

9. A compound according to claim 1 wherein R^{a} and R^{b} are hydrogen.

10. A compound according to claim 1 wherein R^{c} is hydrogen.

11. A compound according to claim 1 wherein R³ is arylalkyl.

12. A compound of formula 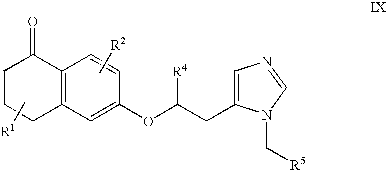
and pharmaceutically acceptable salts, esters, amides, and prodrugs thereof,
wherein:
R² is hydrogen, (C₁-C₆)-alkyl, aryl, heteroaryl, arylalkyl, or heteroarylalkyl, wherein the aryl, heteroaryl, arylalkyl, or heteroarylalkyl is optionally substituted with a group independently selected from the group consisting of alkyl, O-alkyl, S-alkyl, OH, SH, —CN, halogen, 1,3-dioxolanyl, CF₃, NO₂, NH₂, NHCH₃, N(CH₃)₂, NHCO-alkyl, —(CH₂)ₘCO₂H, —(CH₂)ₘCO₂-alkyl, —(CH₂)ₘSO₃H, —NH-alkyl, —N(alkyl)₂, —(CH₂)ₘPO₃H₂, —(CH₂)ₘPO₃(alkyl)₂, —(CH₂)ₘSO₂NH₂, —(CH₂)ₘ-heteroaryl, —(CH₂)ₘS-aryl, —(CH₂)ₘS-heteroaryl, —(CH₂)ₘSO₂-aryl, —(CH₂)ₘSO₂-heteroaryl, and —(CH₂)ₘSO₂NH-alkyl, wherein m is 0, 1, 2, or 3, and wherein each of the R¹ and R² groups can be attached through a linker, or through a lower alkyl optionally interrupted by a linker, said linker selected from the group consisting of 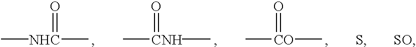
SO₂, O, and NH;
R⁴ is hydrogen or phenyl; and
R⁵ is aryl optionally substituted by (C₁-C₆)-alkyl, (C₁-C₆)-alkoxy, or cyano.

13. A compound of formula 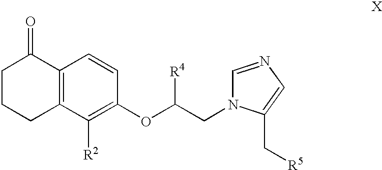
and pharmaceutically acceptable salts, esters, amides, and prodrugs thereof,
wherein:
R² is hydrogen, (C₁-C₆)-alkyl, aryl, heteroaryl, arylalkyl, or heteroarylalkyl, wherein the aryl, heteroaryl, arylalkyl, or heteroarylalkyl is optionally substituted with a group independently selected from the group consisting of alkyl, O-alkyl, S-alkyl, OH, SH, —CN, halogen, 1,3-dioxolanyl, CF₃, NO₂, NH₂, NHCH₃, N(CH₃)₂, NHCO-alkyl, —(CH₂)ₘCO₂H, —(CH₂)ₘCO₂-alkyl, —(CH₂)ₘSO₃H, —NH-alkyl, —N(alkyl)₂, —(CH₂)ₘPO₃H₂, —(CH₂)ₘPO₃ (alkyl)₂, —(CH₂)ₘSO₂NH₂, —(CH₂)ₘ-heteroaryl, —(CH₂)ₘS-aryl, —(CH₂)ₘS-heteroaryl, —(CH₂)ₘSO₂-aryl, —(CH₂)ₘSO₂-heteroaryl, and —(CH₂)ₘSO₂NH-alkyl, wherein m is 0, 1, 2, or 3, and wherein each of the R¹ and R² groups can be attached through a linker, or through a lower alkyl optionally interrupted by a linker, said linker selected from the group consisting of 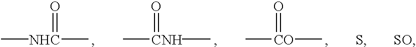
SO₂, O, and NH;
R⁴ is hydrogen or phenyl; and
R⁵ is aryl optionally substituted by (C₁-C₆)-alkyl, (C₁-C₆)-alkoxy, or cyano.

14. A compound of claim 12 or 13 wherein R² is hydrogen, (C₁-C₆)-alkyl, aryl, heteroaryl, arylalkyl, or heteroarylalkyl.

15. A compound of claim 14 wherein the arylalkyl is substituted with —(CH₂)ₘCO₂H.

16. A compound of claim 12 or 13 wherein the linker is selected from the group consisting of —NHCO, —CO₂, SO₂, O, and —NH.

17. A compound of claim 16 wherein R² is (C₁-C₆)-alkyl, aryl, or heteroaryl.

18. A compound of claim 12 or 13 wherein R⁴ is hydrogen.

19. A compound according to claim 1, which is selected from the group consisting of
4-{5-[2-(5-oxo-5,6,7,8-tetrahydronaphthalen-2-yloxy)ethyl] imidazol-1-ylmethyl}benzonitrile;
4-{5-[2-(5-oxo-1-phenethyl-5,6,7,8-tetrahydronaphthalen-2-yloxy)ethyl]imidazol-1-ylmethyl}benzonitrile;
4-(2-{2-[2-(3-benzyl-3H-imidazol-4-yl)ethoxy]-5-oxo-5,6,7,8-tetrahydronaphthalen-1-yl} ethyl)benzoic acid;
6-[2-(3-benzyl-3H-imidazol-4-yl)ethoxy]-5-phenylaminomethyl-3,4-dihydro-2H-naphthalene-1-one;
5-benzyl-6-[2-(3-benzyl-3H-imidazol-4-yl)ethoxy]-3,4-dihydro-2H-naphthalene-1-one;
6-[2-(3-benzyl-3H-imidazol-4-yl)ethoxy]-5-phenylamino-3,4-dihydro-2H-naphthalene-1-one;
N-{2-[2-(3-benzyl-3H-imidazol-4-yl)ethoxy]-5-oxo-5,6,7,8-tetrahydro-naphthalene-1-yl} benzamide;
6-[2-(3-benzyl-3H-imidazol-4-yl)ethoxy]-5-isopropoxymethyl-3,4-dihydro-2H-naphthalene-1-one;
3-{2-[2-(3-benzyl-3H-imidazol-4-yl)ethoxy]-5-oxo-5,6,7,8-tetrahydro-naphthalene-1-yl}propionic acid methyl ester;
6-[2-(3-benzyl-3H-imidazol-4-yl)-1-phenylethoxy]-3,4-dihydro-2H-naphthalene-1-one;
4-{3-[2-(5-oxo-5,6,7,8-tetrahydronaphthalen-2-yloxy)ethyl]-3H-imidazol-4-yl}methyl)benzonitrile;
6-[2-(5-benzyl-imidazol-1-yl)ethoxy]-5-propyl-3,4-dihydro-2H-naphthalene-1-one;
6-[2-(5-benzyl-imidazol-1-yl)ethoxy]-5-phenethyl-3,4-dihydro-2H-naphthalene-1-one;
4-{3-[2-(5-oxo-1-phenethyl-5,6,7,8-tetrahydronaphthalen-2-yloxy)ethyl]-3H-imidazol-4-yl}methyl)benzonitrile;
4-(2-{2-[2-(5-benzyl-imidazol-1-yl)ethoxy]-5-oxo-5,6,7,8-tetrahydronaphthalen-1-yl} ethyl)benzoic acid;
6-[2-(5-benzyl-imidazol-1-yl)ethoxy]-5-phenylaminomethyl-3,4-dihydro-2H-naphthalene-1-one;
5-benzyl-6-[2-(5-benzyl-imidazol-1-yl)ethoxy]-3,4-dihydro-2H-naphthalene-1-one;
6-[2-(5-benzyl-imidazol-1-yl)ethoxy]-5-phenylamino-3,4-dihydro-2H-naphthalene-1-one;
N-{2-[2-(5-benzyl-imidazol-1-yl)ethoxy]-5-oxo-5,6,7,8-tetrahydro-naphthalene-1-yl}benzamide;
6-{2-[3-(methoxy-3-methylbenzyl)-3H-imidazol-4-yl]ethoxy}-5-phenethyl-3,4-dihydro-2H-naphthalene-1-one;
6-{2-[3-(4-methoxy-3-methyl-benzyl)-3H-imidazol-4-yl]-ethoxy}-3,4-dihydro-2H-naphthalen-1-one;
6-[2-(3-benzyl-3H-imidazol-4-yl)-ethoxy]-5-propyl-3,4-dihydro-2H-naphthalen-1-one;
6-[2-(3-Benzyl-3H-imidazol-4-yl)-ethoxy]-5-phenethyl-3,4-dihydro-2H-naphthalen-1-one
6-[2-(5-Benzyl-3H-imidazol-1-yl)-ethoxy]-5-(2-pyridin-2-yl-ethyl)-3,4-dihydro-2H-naphthalene-1-one;
6-{2-[3-(4-Methoxy-3-methylbenzyl)-3H-imidazol-4-yl]ethoxy}-5-(2-pyridin-2-ylethyl)-3,4-dihydro-2H-naphthalene-1-one;
5-Benzenesulfonylmethyl-6-{2-[5-(4-methoxy-3-methylbenzyl)imidazol-1-yl]ethoxy}-3,4-dihydro-2H-naphthalene-1-one;
5-Benzenesulfonylmethyl-6-{2-[3-(4-methoxy-3-methylbenzyl)-3H-imidazol-4-yl]ethoxy}-3,4-dihydro-2H-naphthalene-1-one;
4-({5-[2-({5-Oxo-1-[(2-pyridinylsulfonyl)methyl]-5,6,7,8-tetrahydro-2-naphthalenyl} oxy)ethyl]-1H-imidazol-1-yl}methyl)-benzonitrile; and
4-({5-[2-({1 [(Isopropylsulfonyl)methyl]-5-oxo-5,6,7,8-tetrahydro-2-naphthalenyl} oxy)ethyl]-1H-imidazol-1-yl}methyl)benzonitrile.

20. A compound of claim 1, which is 4-({5-[2-({5-Oxo-1-[(2-pyridinylsulfonyl)methyl]-5,6,7,8-tetrahydro-2-naphthalenyl}oxy)ethyl]-1H-imidazol-1-yl}methyl)-benzonitrile or 4-({5-[2-({1[(Isopropylsulfonyl)methyl]-5-oxo-5,6,7,8-tetrahydro-2-naphthalenyl} oxy)ethyl]-1H-imidazol-1-yl} methyl)benzonitrile.

21. A pharmaceutical composition comprising a compound of claim 1 and a pharmaceutically acceptable carrier, excipient, or diluent.

22. A method of treating or preventing restenosis or atherosclerosis, the method comprising administering to a patient having restenosis or atherosclerosis or at risk of having restenosis or atherosclerosis a therapeutically effective amount of a compound of claim 1.

23. A method of treating cancer, the method comprising administering to a patient having cancer a therapeutically effective amount of a compound of claim 1.
